(19) United States
(12) Patent Application Publication
(10) Pub. No.: US 2007/0053954 A1
(43) Pub. Date: Mar. 8, 2007

(54) Macromer-melt formulations

(76) Inventors: Stephen C. Rowe, Wellesley, MA (US); Durga Annavajjula, Waltham, MA (US)

Correspondence Address: CLARK & ELBING LLP, 101 FEDERAL STREET, BOSTON, MA, 02110, US

(21) Appl. No.: 11/410,269

(22) Filed: Apr. 24, 2006

Related U.S. Application Data
(63) Continuation-in-part of application No. PCT/US04/35346, filed on Oct. 22, 2004.
(63) Continuation-in-part of application No. PCT/US04/35088, filed on Oct. 25, 2004.
(63) Continuation-in-part of application No. PCT/US04/35267, filed on Oct. 22, 2004.
(60) Provisional application No. 60/514,286, filed on Oct. 24, 2003.
(60) Provisional application No. 60/514,243, filed on Oct. 24, 2003.
(60) Provisional application No. 60/514,292, filed on Oct. 24, 2003.

(51) Int. Cl. A61F 2/02

(52) U.S. Cl. 424/426

(57) ABSTRACT

The invention provides methods and articles for the administration of a biologically active substance (BAS). These methods and articles provide for the controlled and sustained delivery of relatively large quantities of these substances with a low burst effect. The articles made using the method of the invention have increased percentages (w/w) of macromer, increased crosslinking density, and reduced pore size in comparison to articles made using solution methods.

### CROSS-REFERENCE TO RELATED APPLICATIONS

This application is a Continuation-In-Part of PCT Application No. PCT/US04/35346, filed Oct. 22, 2004, which claims the benefit of U.S. Provisional Application No. 60/514,286, filed Oct. 24, 2003, and this application is also a Continuation-In-Part of PCT Application No. PCT/US04/35088, filed Oct. 25, 2004, which claims the benefit of U.S. Provisional Application No. 60/514,243, filed Oct. 24, 2003, and this application is also a Continuation-In-Part of PCT Application No. PCT/US04/35267, filed Oct. 22, 2004, which claims the benefit of U.S. Provisional Application No. 60/514,292, filed Oct. 24, 2003. Each of the above-referenced applications are incorporated herein by reference.

### BACKGROUND OF THE INVENTION

The invention relates to biodegradable articles for sustained-release drug delivery and methods for administering a biologically active substance via these articles.

The rapid advances in the fields of genetic engineering and biotechnology have led to the development of an increasing number of proteins and peptides that are useful as pharmaceutical agents. The development of methods for administering these new pharmaceutical agents is thus becoming increasingly important. However, these molecules are generally limited to parenteral administration due to their susceptibility to degradation in the gastrointestinal tract. Treatment for chronic illnesses or indications may require multiple injections per day or injections several times per week over extended periods of time. As a result of the need for frequent injections, patient compliance may be less than optimal.

Attempts to maintain a steady level of medication in the blood stream using biodegradable polymer vehicles has attracted considerable attention. These vehicles are biodegradable and do not require retrieval after the medication is exhausted. Therefore, they can be fabricated into microspheres, microcapsules, nanospheres, implantable rods, or other physical shapes with the drug encapsulated within.

A burst release of the agent is often observed immediately after administration of the biodegradable delivery system, especially for low molecular weight agents. Burst is often a problem where the primary mechanism of drug release from the biodegradable polymer is diffusion. The initial burst results in much higher than normal therapeutic levels of medication in the blood. These high levels of agent can cause side effects such as nausea, vomiting, delirium and, sometimes, death.

Therefore, it would be desirable to identify both a delivery vehicle and method of encapsulating a biologically active agent therewith that results in little or no burst release of the agent.

### SUMMARY OF THE INVENTION

The present invention features articles for delivery of a biologically active substance (hereafter “BAS”), and methods for making such articles. The articles made using the method of the invention have increased percentages (w/w) of macromer, increased crosslinking density, and reduced pore size in comparison to articles made using solution methods. The articles exhibit extended release profiles, even for low molecular weight active substances. The invention also features methods of treating a mammal using the articles described herein.

Accordingly, in a first aspect the invention features a therapeutic article for delivery of a BAS, including a BAS within a polymerized macromer, the macromer including at least one water soluble polymer region, at least one degradable polymer region which is hydrolyzable under in vivo conditions, and polymerized end groups, wherein the polymerized end groups are separated by at least one degradable polymer region. When fully hydrated the article includes at least 35% (w/w) polymerized macromer. Desirably, the fully hydrated article includes at least 40%, 45%, 50%, 55%, 60%, 65%, 70%, 75%, 80%, 85%, 90%, or even 95% (w/w) polymerized macromer.

In a preferred embodiment of the first aspect of the invention, the article when fully hydrated includes less than 50% (w/w) water. Desirably, the fully hydrated article includes less than 45%, 40%, 35%, 30%, 25%, 20%, 15%, or even 12% (w/w) water.

In a second aspect, the invention features a method for making a controlled release therapeutic article for delivery of a BAS, wherein the article includes a BAS within a polymerized macromer, the macromer including at least one water soluble polymer region, at least one degradable polymer region which is hydrolyzable under in vivo conditions, and polymerized end groups, wherein the polymerized end groups are separated by at least one degradable polymer region. The method includes the steps of: a) heating the macromer until it melts; b) forming a mixture of biologically active substance and melted macromer; and c) polymerizing the mixture to form the therapeutic article.

In one embodiment of the second aspect of the invention, the mixture of step (b) is emulsified prior to step (c). The emulsion can be formed with a non-miscible continuous phase liquid (e.g., propylene glycol, mineral oil). Alternatively, the mixture of step (b) can be sprayed from a nozzle to produce small droplets, which are then polymerized, for example, upon exposure to UV light.

In another embodiment of the second aspect, the mixture of step (b) comprises a biologically active substance in the form of a particle having a mean particle size of 0.02 to 10 microns. Desirably, the biologically active substance in the form of a particle having a mean particle size of 0.02 to 5 microns, 0.05 to 10 microns, 0.05 to 5 microns, 0.1 to 5 microns, or 0.02 to 0.5 microns.

In a preferred embodiment of the second aspect of the invention, the article when fully hydrated includes at least 35% (w/w) polymerized macromer. Desirably, the fully hydrated article includes at least 40%, 45%, 50%, 55%, 60%, 65%, 70%, 75%, 80%, 85%, 90%, or even 95% (w/w) polymerized macromer.

In another preferred embodiment of the second aspect of the invention, the article when fully hydrated includes less than 50% (w/w) water. Desirably, the fully hydrated article includes less than 45%, 40%, 35%, 30%, 25%, 20%, 15%, or even 12% (w/w) water.

In a third aspect, the invention features a method of treating a mammal including administering a therapeutic article of the first aspect of the invention to a mammal. Desirably, the mammal is a dog, cat, cow, pig, horse, sheep, goat, or human.

In yet other embodiments of the third aspect, the articles are administered systemically or locally. Desirably, the articles are administered to the lung of the mammal, or are administered subcutaneously, intramuscularly, intravenously, orally, nasally, or locally at the site of disease. Examples of local administration include, without limitation, ocular administration to treat eye disease or intra-tumor administration to treat cancer.

In an embodiment of any of the above aspects, the BAS has a molecular weight of less than about 30,000 Daltons. Desirably, the molecular weight of the BAS is less than 25,000, 20,000, 15,000, 10,000, 7,000, 5,000, 3,000 or even 1,500 Daltons.

In another embodiment of any of the above aspects, the polymerized macromer includes: (a) a region forming a central core; (b) at least two degradable regions attached to the core; and (c) at least two polymerized end groups, where the polymerized end groups are attached to the degradable regions.

Desirably, the region forming a central core is a water soluble region. The water soluble region may be poly(ethylene glycol), poly(ethylene oxide), poly(vinyl alcohol), poly(vinylpyrrolidone), poly(ethyloxazoline), poly(ethylene oxide)-co-poly(propylene oxide) block copolymers, polysaccharides, carbohydrates, proteins, and combinations thereof. For example, the water soluble region may consist essentially of PEG having a molecular weight of about 500 to 30,000 daltons, or more preferably, between 1,000 and 10,000 daltons.

Degradable regions include, without limitation, poly(α-hydroxy acids), poly(lactones), poly(amino acids), poly(anhydrides), poly(orthoesters), poly(orthocarbonates), poly(α-hydroxy alkanoates), poly(dioxanones), and poly(phosphoesters). The poly(α-hydroxy acid) can be poly(glycolic acid), poly(DL-lactic acid), or poly(L-lactic acid), and the poly(lactone) is poly(ε-caprolactone), poly(δ-valerolactone), or poly(γ-butyrolactone). Desirably, the degradable region includes poly(caprolactone). The degradable region may include a blend of at least two different polymers.

Desirably, the polymerizable end groups contain a carbon-carbon double bond capable of polymerizing the macromer.

In another embodiments of any of the above aspects, the macromer includes: (a) a water soluble region including a three-armed poly(ethylene glycol); (b) lactate groups attached to the region in (a); and (c) acrylate groups capping the region in (b). The macromer may alternatively include: (a) a water soluble region including a three-armed poly(ethylene glycol); (b) lactate groups on either side of the region in (a); and (c) acrylate groups capping either side of the region in (b). In another alternative, the macromer may include (a) a water soluble region including a three-armed poly(ethylene glycol); (b) caprolactone groups on either side of region in (a); and (c) acrylate groups capping either side of the region in (b).

In one embodiment of any of the above aspects, the macromer includes a water soluble region consisting of a three-armed, four-armed, five-armed, six-armed, seven-armed, or eight-armed PEG with a molecular weight of 1,000 to 20,000, 1,000 to 15,000, 1,000 to 10,000, 1,000 to 7,000, 2,000 to 6,000, 4,200 to 5,400 daltons; degradable polymers at the end of each arm of the PEG; and polymerizable end groups attached to each of the degradable polymers.

In another embodiment of any of the above aspects, the macromer includes a water soluble region consisting of a three-armed PEG with a molecular weight of 4,200 to 5,400 daltons; lactate groups one end of each arm of the PEG; and acrylate groups capping the lactate groups. The macromer can also be made of a triad ABA block copolymer of acrylate-poly(lactic acid)-PEG-acrylate-poly(lactic acid)-acrylate. The PEG has a MW of 3,400 daltons; the poly(lactic acids) on both sides have an average of about five lactate units per side; and the macromer is therefore referred to herein as A3.4kL5. A lower molecular weight PEG, such as MW 2,000 daltons PEG can be used in place of the MW 3,400 PEG, and the resulting macromer is abbreviated as “2kL5.” The macromer is an acrylate-PCL-PEG-PCL-acrylate macromer. The PEG has a MW of 3,400 daltons and has polycaprolactone (PCL) on both sides, with an average of about 6 caproyl units per side. This macromer is referred to herein as “3.4kC6.”

In still other embodiments of any of the above aspects, the article includes at least 0.1% BAS by dry weight. Desirably, the article includes at least 0.5%, 1%, 2%, 3%, 4%, 5%, 10%, 15%, 20%, 25%, or even 30% BAS by dry weight.

In an embodiment of any of the above aspects, the BAS is selected from peptides, carbohydrates, inorganic materials, antibiotics, antineoplastic agents, local anesthetics, antiangiogenic agents, vasoactive agents, anticoagulants, RNAi, antisense oligonucleotides, immunomodulators, cytotoxic agents, antiviral agents, antibodies, neurotransmitters, psychoactive drugs, oligonucleotides, proteins, lipids, and combinations thereof.

In one embodiment, the BAS is a peptide. Peptides which can be used in the articles and methods of the invention include, without limitation, Acetelins, ACTH Peptides, Adrenomedullins, Amylins, Anti-HIV peptides, Anti-Inflammatory Peptides, Anti-Oxidant Peptides, Angiotensins, Apelins, BAM Peptides, Basic Fibroblast Growth Factor (FGF) Inhibitory Peptides, Bombesins, Bradykinins, Bradykinin-Potentiating Peptides (BPP), C3a and C3d Peptides, C5a-Related Peptides, Caerulein, Calcitonin and Calcitonin Precursors, Calcitonin Gene-Related Peptides (CGRP), Calpain Inhibitors, α-Casein Exorphins, β-Casomorphins, Cathepsin G Peptides, Cecropins, Ceratotoxins, Cerebellins, Cholecystokinin-Pancreozymin Peptides, Chorionic Gonadotropin (hCG) Peptides, CKS-17, Cocaine and Amphetamine Regulated Transcript (CART) Peptides, Conantokin G peptides, Corticotropin-Releasing Factor (CRF) and Analogs, C-Reactive Protein (CRP) Sequences, Defensins, Delta-Sleep Inducing Peptides (DSIP), Deltorphins, and Dermorphins, Eglin c peptides, Endomorphins, Endorphins, Endothelin Antagonists, Enkephalins and Proenkephalins, Farnesyltransferase Inhibitors, FIV Peptide, FMRFamide Peptides, Galanins and Galanin Message Associated Peptides (GMAP), Gastrins, Gastrin Releasing Peptides (GRP), Ghrelins, Glucagons and Glucagon-Like Peptides, Gluten Exorphins, GM-CSF Inhibitory Peptides, Growth Hormone-Releasing Factors (GRF) and Peptides (GHRP), Helodermins, Hirudins, Hylambatins, Insulin-like growth factors (IGF), Interleukins, Kinetensin s, Kyotorphins, Laminins, Leptins, Leucokinins, Leupeptins, Luteinizing hormone-releasing Hormone Peptides, Mastoparans, Melanin-Concentrating Hormones (MCH), Melanocyte-Stimulating Hormone-Release Inhibiting Factors (MIF-I), Melanotropin-Potentiating Factors (WPF), Motilins, Melanin—Stimulating Hormone (MSH) Peptides, Morphine Modulating Neuropeptides, Natriuretic Peptides and Related Peptides, Neoendorphins, Neurokinins, Neuromedins, Neuropeptide Y (NPY), Neurotensins, Nociceptins, Orexins, Oxytocins, Pancreatic Polypeptides, Peptide YY (PYY), Pituitary Adenylate Cyclase Activating Polypeptides (PACAP), Pneumadins, Prolactin-Releasing Peptides, Protein Kinase Related Peptides, Protein Kinase Related Peptides, Secretins, Somatostatins, Substance P, Syndyphalins, Thymopoietins, Thymosins, Thyrotropin—Releasing Hormone (TRH), Tuftsins, Urocortins, Valorphins, Vasopressins, Vasoactive intestinal peptides (VIP), collagenase-1 inhibitors, stromelysin-1 inhibitors, erythropoietin peptide agonists, follicle stimulating hormone antagonists, human neutrophil elastase inhibitors, kallikrein inhibitors, selectin binding peptides, exendins, exendin-4, and analogs thereof.

In a related embodiment, the peptide is an opioid peptide. Opioid peptides include, without limitation, Acetalins, BAM Peptides, α-Casein Exorphins, β-Casomorphins, Deltorphins, Dermorphins, Endomorphins, Endorphins, Enkephalins, Gluten Exorphins, Kyotorphins, Metorphamide, Neoendorphins, Syndyphalins, Valorphins, and analogs thereof.

In another related embodiment, the peptide is an antimicrobial peptide. Antimicrobial peptides include, without limitation, Cathepsin G Peptides, Cecropins, Ceratotoxins, Defensins, and analogs thereof.

Desirably the peptide is selected from Antide, Buserelin, Deslorelin, Fertirelin, Gonadorelin, Goserelin, Histrelin, Leuprolide, Nafarelin, Triptorelin, Calcitonin, Elcatonin, Corticotropin-Releasing Factor, Glucagon (1-29), Glucagon—Like Peptide-1 (7-37), GRF (1-29) Amide, Growth Hormone-Releasing Factor, Insulin, Octreotide, Somatostatin-14, Thymalfasin, Thymosin β4, Desmopressin, Dynorphin A (1-13), Oxytocin, Protirelin, Secretin, Sincalide, Thymopentin, Vasoactive Intestinal Peptide, exendins, exendin-4, and analogs thereof. Peptides that can be used in accordance with the invention include the peptides listed in Table 1 and any other peptide described herein or an analog thereof.

In another embodiment, the BAS is a protein. Proteins which can be used in the articles and methods of the invention include, without limitation, growth hormones, such as human growth hormone and bovine growth hormone; enzymes, such as DNase, proteases, urate oxidase, alronidase, alpha galactosidase, and alpha glucosidase; antibodies, such as trastuzumab (Genentech),. oprelvekin (Genetics Institute), muromonab-CD3 (Ortho Biotech), infliximab (Centocor), abciximab (Eli Lilly), ritiximab (Genentech), basiliximab (Novartis), palivizumab (MedImmune), thymocyte globulin (SangStat), cetuximab (ImClone), and daclizumab (Hoffman-La Roche); poetins, such as erythropoietin and thrombopoietin; cytokines, such as TNF-alpha; interferons, such as interferon alpha and interferon beta; angiogenic factors; growth factors, including vascular endothelial growth factor (VEGF), endothelial cell growth factor (ECGF), epidermal growth factor (EGF), basic fibroblast growth factor (bFGF), and platelet derived growth factor (PDGF); clotting factors, such as factor IV, factor VIII, and factor VIIa; thyrotropin alfa; tissue plasminogen activator; glucocere-brosidase; etanercept (Immunex, Amgen); pegademase bovine (Enzon); colony stimulating factor (GMCSF); follicle-stimulating hormone (FSH); luteinizing hormone (LH); prolactin; relaxin; somatotropin-releasing hormones; tachykinins; thyroid-stimulating hormone (TSH); differentiation factors; colony-stimulating factors; ceredase; gibberellins; auxins; rhIGF-I/rhIGFBP-3 complex, and analogs thereof.

In an embodiment of any of the above aspects, the time at which 5% of the releasable BAS is released from the article is greater than 1/16 of t₅₀. The articles of the invention can release BAS such that t₅₀ is greater than or equal to ⅝ of t₈₀. The therapeutic articles of the invention can be capable of releasing the BAS for at for a period of time at least 2 times greater than t₅₀. The article can also capable of delivering a therapeutic dose of the BAS for at for a period of time at least 11/4 times greater than t₅₀.

In yet another embodiment of any of the above aspects, at least 80% of the therapeutic articles may have a particle size of less than about 80 microns. Desirably, at least 80% of the therapeutic articles have a particle size of less than 50, 40, 30, 20, 10, 5, 4, 3, 2, 1, or even 0.5 microns.

The density of the particles is expressed in terms of tap density. Tap density is a standard measure of the envelope mass density. The envelope mass density of an isotropic particle is defined as the mass of the particle divided by the minimum sphere envelope volume within which it can be enclosed. The density of particles can be measured using a GeoPyc (micrometers Instrument Corp., Norcross, Ga.) or a AutoTap (Quantachrome Corp., Boyton Beach, Fla.).

In one embodiment of the first and second aspects of the invention, the tap density of the articles is greater than 0.6 g/cm³. Desirably, the tap density is greater than 0.65, 0.70, 0.75, 0.80, 0.85, 0.90, 0.95, 1.1, 1.2, 1.3, 1.4, or even 1.5 g/cm³.

In one embodiment of any of the above aspects, the therapeutic article is biocompatible.

In another embodiment of any of the above aspects, the degradable polymer region is hydrolyzed in the presence of water.

In yet another embodiment of any of the above aspects, the degradable polymer region is hydrolyzed enzymatically.

The methods and compositions described herein can also be used to generate information useful, for example, for increasing investment in a company or increasing consumer demand for the methods and/or compositions.

The invention therefore features a method of increasing consumer demand for a pharmaceutical composition (e.g., the articles of the invention) or therapeutic regimen (e.g., the administration of articles of the invention) described herein. The method includes the step of disseminating information about the pharmaceutical composition or therapeutic regimen.

The invention further features a method of increasing investment in a company seeking governmental approval for the sale of a pharmaceutical composition and/or therapeutic regimen described herein. The method includes the steps of i) disseminating information about the pharmaceutical composition or therapeutic regimen and ii) disseminating information about the intent of the company to market the pharmaceutical composition or therapeutic regimen.

Consumer demand for a pharmaceutical composition described herein can be increased by disseminating information about the utility, efficacy, or safety of the pharmaceutical composition. Consumers include health maintenance organizations, hospitals, doctors, and patients. Typically, the information will be disseminated prior to a governmental approval for the sale of a composition or therapeutic regimen of the invention.

A company planning to sell a pharmaceutical composition described herein can increase investment therein by disseminating information about the company's intention to seek governmental approval for the sale of and disseminating information about the pharmaceutical composition and/or therapeutic regimen of the invention. For example, the company can increase investment by disseminating information about in vivo studies conducted, or planned, by the company, including, without limitation, information about the toxicity, efficacy, or dosing requirements of a pharmaceutical composition or therapeutic regimen of the invention. The company can also increase investment by disseminating information about the projected date of governmental approval of a pharmaceutical composition or therapeutic regimen of the invention.

Information can be disseminated in any of a variety of ways, including, without limitation, by press release, public presentation (e.g., an oral or poster presentation at a trade show or convention), on-line posting at a web site, and mailing. Information about the pharmaceutical composition or therapeutic regimen can include, without limitation, a structure, diagram, figure, chemical name, common name, tradename, formula, reference label, or any other identifier that conveys the identity of the pharmaceutical composition or therapeutic regimen of the invention to a person.

By “in vivo studies” is meant any study in which a pharmaceutical composition or therapeutic regimen of the invention is administered to a mammal, including, without limitation, non-clinical studies, e.g., to collect data concerning toxicity and efficacy, and clinical studies.

By “projected date of governmental approval” is meant any estimate of the date on which a company will receive approval from a governmental agency to sell, e.g., to patients, doctors, or hospitals, a pharmaceutical composition or therapeutic regimen of the invention. A governmental approval includes, for example, the approval of a drug application by the Food and Drug Administration, among others.

As used herein, “analog” refers to a peptide or protein incorporated as a BAS into an article of the invention. The present invention is applicable to analogs of any peptide or protein described herein. An analog is any substitution, rearrangement, deletion, truncation, addition, or combination thereof to the amino acid sequence of a peptide or protein described herein, so long as the peptide or protein and corresponding analog share the same therapeutic activity. Analogs also include peptides or proteins which contain additional amino acids or capping groups added to either terminus of the sequence provided that the therapeutic activity of the peptide or protein is retained. An algorithm can be used in the identification of analogs, such as the BLASTP program (Altschul, J. Mol. Evol. 36:290 (1993); Altschul, J. Mol. Biol. 215:403 (1990)). The amino acid sequence of the analog shares at least 70% homology with the peptide or protein recited herein. In a preferred embodiment, the peptide or protein and analog are at least 75%, 80%, 85%, 90%, or 95% homologous.

By “macromer” is meant a polymer with three components: (1) a biocompatible, water soluble region; (2) a degradable region, and (3) at least two polymerizable regions.

By “biologically active substance” or “BAS” is meant a compound, be it naturally-occurring or artificially-derived, that is incorporated into an article and which may be released and delivered to a site. Biologically active substances may include, for example, peptides, proteins, synthetic organic molecules, naturally occurring organic molecules, nucleic acid molecules, and components thereof.

By “biocompatible” is meant that any compound or substance which is administered to a subject, cell, or tissue is used to treat, replace, or augment a function of the subject, cell or tissue, and is not harmful to the function. Biocompatible substances and compounds produce minimal immune cell infiltration and encapsulation when injected in vivo. As a result, the bioavailability of the BAS is not reduced by immunological responses.

As used herein, “hydrolyzable under in vivo conditions” refers to the degradable region of a macromer or therapeutic article. One or more bonds within the degradable region are cleaved by the addition of water. The degradable region can be selected to hydrolytically degrade in aqueous environments. Examples of degradable regions that hydrolyze in the presence of water include esters and carbonates, among others. Alternatively, the degradable region can be selected to selectively hydrolyze in the presence of an enzyme. Examples of degradable regions that can be enzymatically hydrolyzed in vivo include polypeptides, among others.

As used herein, “exendin-4” refers to the peptide of SEQ ID NO. 1: His Gly Glu Gly Thr Phe Thr Ser Asp Leu Ser Lys Gln Met Glu Glu Glu Ala Val Arg Leu Phe Ile Glu Trp Leu Lys Asn Gly Gly Pro Ser Ser Gly Ala Pro Pro Pro Ser-NH₂.

As used herein, “exendin” and “exendins” refers to an insulinotropic peptide of formula (I) or analog thereof.

| | | |
|---|---|---|
| Xaa¹-Xaa²-Xaa³-Gly-Thr- | Formula (1) | (SEQ ID NO:1) |
| Phe-Thr-Xaa⁸-Xaa⁹-Xaa¹⁰- | | |
| Ser-Xaa¹²-Xaa¹³-Xaa¹⁴-Glu- | | |
| Xaa¹⁶-Xaa¹⁷-Ala-Xaa¹⁹- | | |
| Xaa²⁰-Xaa²¹-Phe-Ile-Xaa²⁴- | | |
| Xaa²⁵-Leu-Xaa²⁷-Xaa²⁸-Gly- | | |
| Xaa³⁰-R³¹ | | |

In formula (I), Xaa¹ is selected from L-histidine, D-histidine, desaminohistidine, 2-amino-histidine, β-hydroxyhistidine, homohistidine, α-fluoromethyl-histidine, and α-methyl-histidine; Xaa² is selected from glycine, alanine, serine, and valine; Xaa³ is selected from aspartic acid and glutamic acid; Xaa⁸ is selected from serine and glutamic acid; Xaa⁹ is selected from aspartic acid and glutamic acid; Xaa¹⁰ is selected from leucine and valine; Xaa¹² is selected from lysine and serine; Xaa¹³ is selected from glutamine and tyrosine; Xaa¹⁴ is selected from methionine and leucine; Xaa¹⁶ is selected from glutamic acid and glutamine; Xaa¹⁷ is selected from glutamic acid and glutamine; Xaa¹⁹ is selected from valine and alanine; Xaa²⁰ is selected from arginine and lysine; Xaa²¹ is selected from leucine and glutamic acid; Xaa²⁴ is selected from glutamic acid and alanine; Xaa²⁵ is selected from phenylalanine and tryptophan; Xaa²⁷ is selected from valine and lysine; Xaa²⁸ is selected from asparagine and lysine; Xaa³⁰ is selected from glycine and arginine; and R³¹ is selected from glycine, proline, tyrosine, Pro-Ser-Ser-Gly-Ala-Pro-Pro-Pro-Ser, or is absent.

Further included in the definition of exendin is Helodermin (Bachem cat. No. H-5696), (Glu^{8,9})-Helodermin (Bachem cat. No. H-5062), exendin-4 (1-30), exendin-4 (1-30) amide, exendin-4 (1-28) amide, (Leu¹⁴,Phe²⁵) exendin-4 amide, (Leu¹⁴,Phe²⁵) exendin-4 (1-28) amide, and ZP10A (Zealand Pharmaceuticals/Aventis; see, for example Thorkildsen et al., J. Pharmacol. Exp. Ther. 307:490-6 (2003)).

Exendins include those analogs described in PCT Publication Nos. WO 03/072195; WO 99/25728; WO 99/25727; WO 98/05351; WO 99/40788; WO 99/07404; and WO 99/43708, each of which is incorporated herein by reference. Exendins also include those analogs described in U.S. Pat. No. 6,528,486, which is incorporated herein by reference.

By “therapeutic dose,” when referring to a BAS, is meant a plasma level between the minimum effective level and the toxic level.

As used herein, “pore size” refers to the dimensions of a space in the intact article through which a BAS potentially can pass. Pore sizes which are created using the melt process of the invention are smaller than the previously reported solution-phase polymerization described in the prior art. As a result, even low molecular weight substances formulated as described herein are released over longer periods of time.

As used herein, “period of release” is meant the length of time it takes for a specified percent of the BAS to be released from an article. The period of release may be assessed, for example, by measuring the time it takes for 10%, 20%, 30%, 40%, 50%, or 80% of the BAS to be released from the article.

By “low burst effect” is meant that the amount of BAS released from an article is released relatively steadily over time, rather than at an initial fast rate, followed by a slower rate. For example, a BAS has a low burst effect (e.g., less than or equal to 20% burst) upon release from an article when the period of release for 5% of the releasable BAS is greater than 1/16 of t₅₀, or when the t₅₀ is greater than or equal to ⅝ of t₈₀. In contrast to a low burst article, a high burst article (e.g., one which rapidly releases 30% of the BAS) might release 5% of its releasable BAS in less than 1/18 of t₅₀ and have a t₅₀ equal to ½ of t₈₀.

A specific example of a low burst product of the present invention is one in which less than 20% of the BAS comes out in the first day for a product designed to release a BAS for 10 days.

By “t₅₀” is meant the time at which 50% of the releasable BAS has been released. Preferably, the articles of the invention release 5% of the releasable BAS at a time which is greater than 1/16 of t₅₀, or the t₅₀ is greater than or equal to ⅝ of the t₈₀.

By “t₈₀” is meant the time at which 80% of the original load of BAS has been released.

As used herein, the term “dry” refers to articles containing less than 10% water by weight. Desirably, the water content of the dry article is less than 5%, 2%, 1%, 0.5%, or less. Articles can be dried using a variety of techniques, such as lyophilization or by exposure to a stream of dry gas.

As used herein, the term “fully hydrated” refers to articles placed in a stirring solution of phosphate buffered saline at 37° C. (pH=7.4) for one hour and isolated by centrifugation.

As used herein, any reference to the trade or chemical name of a drug product is solely a reference to the biologically active substance contained therein. The articles of the invention identified as including the drug by reference to an existing product need not contain any of the inactive ingredients present in the recited drug product.

### BRIEF DESCRIPTION OF THE DRAWINGS

FIG. 1 is a graph depicting the in vitro release of GLP-1 from a therapeutic article prepared as described in Example 1.

FIG. 2A is a graph depicting the in vitro release of LH-RH from a therapeutic article previously washed with 0.1% Sodium Laurate prepared as described in Example 2.

FIG. 2B is a graph depicting the in vitro release of LH-RH from a therapeutic article previously washed with 0.05% Sodium Laurate prepared as described in Example 2.

FIG. 2C is a graph depicting the in vitro release of LH-RH from a therapeutic article previously washed with 0.005% Sodium Laurate prepared as described in Example 2.

FIG. 3 is a graph depicting the in vitro release of fluticasone propionate prepared as described in Example 3.

### DETAILED DESCRIPTION

The invention provides methods and articles for the administration of a biologically active substance (BAS). These methods and articles provide for the controlled, sustained delivery of relatively large quantities of these substances, with a low burst effect. The articles made using the method of the invention have increased percentages (w/w) of macromer, increased crosslinking density, reduced pore size, and decreased swelling in water in comparison to articles made using solution methods. As a result, the articles exhibit extended release profiles for low molecular weight biologically active substances.

Macromers

The macromers of the present invention have at least one water-soluble region, at least one degradable (e.g., hydrolyzable) region, and at least one polymerizable region. The macromers may be water-soluble or water insoluble. These macromers are polymerized to form hydrogels, which are useful for delivering incorporated substances at a controlled rate. Methods of formulating macromers and shaping them into articles are described, for example in WO99/03454, incorporated herein by reference. An important aspect of the macromers is that the polymerizable regions are separated by at least one degradable region. This separation facilitates uniform degradation in vivo.

The ratio between the water-soluble region and the hydrolyzable region of the macromer determines many of the general properties of the macromer. For example, the water solubility of the macromers can be controlled by varying the percentage of the macromer that consists of hydrophobic degradable groups. Accordingly, the macromer can be altered by changing the identity of the degradable groups or the number of degradable groups.

There are several variations of the macromers of the present invention. For example, the polymerizable regions can be attached directly to the degradable regions; alternatively, they can be attached indirectly via water-soluble, non-degradable regions, with the polymerizable regions separated by a degradable region. For example, if the macromer contains a single water-soluble region coupled to a degradable region, one polymerizable region can be attached to the water-soluble region, and the other to the degradable region.

Typically, the water-soluble region forms the central core of the macromer and has at least two degradable regions attached to it. At least two polymerizable regions are attached to the degradable regions so that, upon degradation, the polymerizable regions, particularly in the polymerized gel form, are separated. Alternatively, if the central core of the macromer is formed by a degradable region, at least two water soluble regions can be attached to the core, and polymerizable regions are attached to each water soluble region.

In some instances, the macromer has a water-soluble backbone region, with a degradable region attached to the macromer backbone. At least two polymerizable regions are attached to the degradable regions, such that they are separated upon degradation, resulting in gel product dissolution. The macromer backbone region can be formed of a degradable backbone region having water-soluble regions as branches or grafts attached to the degradable backbone. Two or more polymerizable regions can be attached to the water soluble branches or grafts.

In another variation, the macromer backbone may have multiple arms; e.g., it may be star-shaped or comb-shaped. The backbone may include a water-soluble region, a biodegradable region, or a water-soluble, biodegradable region. The polymerizable regions are attached to this backbone. Again, the polymerizable regions must be separated at some point by a degradable region.

Throughout the specification, the following nomenclature is used to describe the specific macromers of the invention. In three particular examples, a macromer having a water soluble region consisting of PEG with a molecular weight of 4,000 daltons, with 5 lactate groups on either side of this region, capped on either side with acrylate groups, is referred to as “4kL5.” Similarly, a macromer having a water soluble region consisting of PEG with a molecular weight of 3,400 daltons, with 6 caprolactone groups on either side of this region, capped on either side with acrylate groups, is referred to as “3.4kC6.” Likewise, a macromer having a water soluble region consisting of PEG having a molecular weight of 4,400 daltons and 3 arms, each arm containing 3 lactate groups, extending from this region, capped on either side with acrylate groups, is referred to as “4.4kL3-A3.” “4.4kC5-A3” is a macromer having a water soluble region consisting of PEG having a molecular weight of 4,400 daltons and 3 arms, each arm containing 5 caprolactone groups, extending from this region, capped on either side with acrylate groups. “4.4kC4-A3” is a macromer having a water soluble region consisting of PEG having a molecular weight of 4,400 daltons and 3 arms, each arm containing 4 caprolactone groups, extending from this region, capped on either side with acrylate groups. Other macromers may be identified using this same nomenclature.

As mentioned above, one of the ways in which the release properties of the polymerized macromer can be altered is by making changes to the degradable region. The degradable region can contain, for example, polymers of glycolic acid, lactic acid, caprolactone, trimethylene carbonate, or blends or copolymers thereof. As the degradable region increases in hydrophobicity, the polymerized macromer will degrade in water more slowly. A macromer having a degradable region containing 15-20 lactide units can be prepared; this macromer will provide a relatively fast release rate. A macromer with a degradable region containing 6 caprolactone units will provide a relatively slow release rate. A macromer with a degradable region containing a copolymer of 6 caprolactone units, 4 lactide units, and 4 glycolide units will provide a fast release rate, and a macromer with a degradable region containing a copolymer of 3 lactide units and 7 trimethylene carbonate units will provide an intermediate release rate.

The water soluble region of these macromers is preferably PEG. The water soluble region can have multiple arms; for example, it may be star-shaped or comb-shaped, as described, for example in U.S. Pat. No. 5,410,016, incorporated herein by reference. The water soluble region preferably has 3, 4, 6, or 8 arms and a molecular weight of 500 to 20,000, preferably, 1,000 to 10,000 daltons.

Water-Soluble Region

The water soluble region of the macromer may include poly(ethylene glycol), poly(ethylene oxide), poly(vinyl alcohol), poly(vinylpyrrolidone), poly(ethyloxazoline), poly(ethylene oxide)-co-poly(propylene oxide) block copolymers, polysaccharides, carbohydrates, or proteins, or combinations thereof.

The macromer preferably includes a water soluble core region including PEG, as PEG has high hydrophilicity and water solubility, as well as good biocompatibility. The PEG region preferably has a molecular weight of about 400 to about 40,000 daltons, and more preferably has a molecular weight of about 400 to 20,000, 400 to about 15,000 daltons, about 1,000 to about 12,000 daltons, or about 1,000 to about 10,000 daltons.

Degradable Region

The degradable region of the macromer may contain, for example, poly(α-hydroxy acids), poly(lactones), poly(amino acids), poly(anhydrides), poly(orthoesters), poly(orthocarbonates) or poly(phosphoesters), or blends or copolymers of these polymers.

Exemplary poly(α-hydroxy acids) include poly(glycolic acid), poly(DL-lactic acid), and poly(L-lactic acid). Exemplary poly(lactones) include poly(ε-caprolactone), poly(δ-valerolactone), poly(γ-butyrolactone), poly(1,5-dioxepan-2-one), and poly(trimethylene carbonate).

The degradable region may include a blend of at least two different polymers. Examples of copolymers include a copolymer of caprolactone and glycolic acid; and a copolymer of caprolactone and lactic acid.

Polymerizable Region

The polymerizable regions of the macromer preferably contain carbon-carbon double bonds capable of polymerizing the macromers. The choice of an appropriate polymerizable group permits rapid polymerization and gelation. Polymerizable regions containing acrylates are preferred because they can be polymerized using several initiating systems, as discussed below. Examples of acrylates include acrylate, methacrylate, and methyl methacrylate.

Biologically Active Substances

A BAS that can be incorporated into the articles of the invention include therapeutic, diagnostic, and prophylactic agents. They can be naturally occurring compounds, synthetic organic compounds, or inorganic compounds. Substances that can be incorporated into the articles of the invention include proteins, peptides, carbohydrates, inorganic materials, antibiotics, antineoplastic agents, local anesthetics, antiangiogenic agents, vasoactive agents, anticoagulants, immunomodulators, cytotoxic agents, antiviral agents, antibodies, neurotransmitters, psychoactive drugs, oligonucleotides, proteins, lipids, and combinations thereof.

Exemplary therapeutic agents include growth hormone, for example human growth hormone, calcitonin, granulocyte macrophage colony stimulating factor (GMCSF, e.g., filgrastim or pegfilgrastim, a covalent conjugate of recombinant methionyl human G-CSF), ciliary neurotrophic factor, parathyroid hormone, and the cystic fibrosis transmembrane regulator gene. Other specific therapeutic agents include parathyroid hormone-related peptide, somatostatin, testosterone, progesterone, estradiol, nicotine, fentanyl, norethisterone, clonidine, scopolomine, salicylate, salmeterol, formeterol, albeterol, and valium. For example, the BAS can be an antiinflammatory agent, such as an NSAID or corticosteriod.

Drugs for the treatment of pneumonia may be used, including pentamidine isethionate. Drugs for the treatment of pulmonary conditions, such as asthma, may be used, including albuterol sulfate, β-agonists, metaproterenol sulfate, beclomethasone dipropionate, triamcinolone acetamide, budesonide acetonide, ipratropium bromide, flunisolide, cromolyn sodium, ergotamine tartrate, and protein or peptide drugs such as TNF antagonists or interleukin antagonists.

Other therapeutic agents include cancer chemotherapeutic agents, such as cytokines, chemokines, lymphokines, and substantially purified nucleic acids, and vaccines, such as attenuated influenza virus. Substantially purified nucleic acids that can be incorporated include genomic nucleic acid sequences, cDNAs encoding proteins, expression vectors, antisense molecules that bind to complementary nucleic acid sequences to inhibit transcription or translation, and ribozymes. For example, genes for the treatment of diseases such as cystic fibrosis can be administered. Polysaccharides, such as heparin, can also be administered.

Exemplary diagnostic agents include gases and other commercially available imaging agents that are used in positron emission tomography (PET), computer assisted tomography (CAT), single photon emission computerized tomography, X-ray, fluoroscopy, and magnetic resonance imaging (MRI). Suitable materials for use as contrast agents in MRI include gadolinium chelates, as well as iron, magnesium, manganese, copper, and chromium chelates. Examples of materials useful for CAT and X-rays include iodine based materials.

A preferred BAS is a substantially purified peptide or protein. Proteins are generally defined as consisting of 100 amino acid residues or more; peptides are less than 100 amino acid residues. Unless otherwise stated, the term protein, as used herein, refers to both proteins and peptides. The proteins may be produced, for example, by isolation from natural sources, recombinantly, or through peptide synthesis. Examples include growth hormones, such as human growth hormone and bovine growth hormone; enzymes, such as DNase, proteases, urate oxidase, alronidase, alpha galactosidase, and alpha glucosidase; antibodies, such as trastuzumab (Genentech), oprelvekin (Genetics Institute), muromonab-CD3 (Ortho Biotech), infliximab (Centocor), abciximab (Eli Lilly), ritiximab (Genentech), basiliximab (Novartis), palivizumab (MedImmune), thymocyte globulin (SangStat), cetuximab (ImClone), and daclizumab (Hoffman-La Roche); poetins, such as erythropoietin (e.g., epoetin, Amgen) and thrombopoietin; cytokines, such as TNF-alpha; interferons, such as interferon alpha and interferon beta; angiogenic factors; growth factors, including vascular endothelial growth factor (VEGF), endothelial cell growth factor (ECGF), epidermal growth factor (EGF), basic fibroblast growth factor (bFGF), and platelet derived growth factor (PDGF); clotting factors, such as factor IV, factor VIII, and factor VIIa; thyrotropin alfa; tissue plasminogen activator; glucocere-brosidase; etanercept (Immunex); pegademase bovine (Enzon); colony stimulating factor (GMCSF); follicle-stimulating hormone (FSH); luteinizing hormone (LH); prolactin; relaxin; somatotropin-releasing hormones; tachykinins; thyroid-stimulating hormone (TSH); differentiation factors; colony-stimulating factors; ceredase; gibberellins; auxins; rhIGF-I/rhIGFBP-3 (the recombinant protein complex of insulin-like growth factor-I (IGF-I) and its most abundant binding protein, insulin like growth factor binding protein-3 (IGFBP-3)); and analogs thereof. The BAS can be a trinectin, a protein binding scaffold based on a domain of a naturally occurring plasma protein called fibronectin.

Exemplary peptides that can be encapsulated into the articles of the invention include, without limitation, Peptides include adrenocorticotropic hormone (ACT), β-amyloid(1-40), agouti peptide, agouti-related peptide, anaphylatoxins, CASH (Cortical Androgen-Stimulating Hormone), diabetes associated peptide, gliadorphin, insulin, α- & β-lactorphin, g-melanocyte stimulating hormone-like peptide, neuropeptide P, peptide histidine isoleucine (PHI), collagenase-1 and stromelysin-1 inhibitors (including those described in U.S. Pat. Nos. 5,932,579, 5,929,278, and 5,840,698), erythropoietin peptide agonists (including those described in U.S. Pat. Nos. 5,986,047, 5,830,851, 5,773,569), follicle stimulating hormone antagonists (including those described in U.S. Pat. No. 6,426,357), human neutrophil elastase inhibitors (including those described in U.S. Pat. No. 5,663,143, PCT Publication No. WO 03/066824, WO 92/15605, and WO 96/20278, and European Patent No. 1325931A1), kallikrein inhibitors (including those described in U.S. Pat. Nos. 6,333,402, 6,057,287, 5,994,125, and 5,795,865), selectin binding peptides (including those described in U.S. Pat. Nos. 5,728,802, 5,648,458, and 5,643,873), all of the peptides listed in Table 1, and analogs thereof. Exemplary commercially available peptides and their analogs are listed in Table 1, followed by their respective BACHEM catalogue number.

TABLE 1
Peptides

| | |
|---|---|
| Acetelins | Ac-Arg-Phe-Met-Trp-Met-Arg-NH₂ (H-1992) |
| | Ac-Arg-Phe-Met-Trp-Met-Lys-NH₂ (H-1994) |
| | Ac-Arg-Phe-Met-Trp-Met-Thr-NH₂ (H-1996) |
| ACTH Peptides | ACTH (1-4) (H-1125) |
| | ACTH (1-10) (H-1130) |
| | ACTH (1-13) (H-1135) |
| | ACTH (1-14) (H-1140) |
| | Acetyl-ACTH (1-14) (H-1085) |
| | ACTH (1-16) (H-6050) |
| | Acetyl-ACTH (1-17) (H-1090) |
| | ACTH (1-17) (H-1145) |
| | ACTH (1-24) (H-1150) |
| | (D-Lys¹⁶)-ACTH (1-24) (H-4996) |
| | (Phe²,Nle⁴)-ACTH (1-24) (H-6080) |
| | (D-Ser¹)-ACTH (1-24) (H-4718) |
| | ACTH (1-39) (H-1160) |
| | ACTH (3-24) (H-4716) |
| | ACTH (4-9) (H-1165) |
| | (Met(O)⁴,D-Lys⁸,Phe⁹)-ACTH (4-9) (H-1175) |
| | Tyr-ACTH (4-9) (H-1170) |
| | ACTH (4-10) (H-1180) |
| | (p-Iodo-Phe⁷)-ACTH (4-10) (H-2784) |
| | Tyr-ACTH (4-10) (H-1185) |
| | ACTH (4-11) (H-1190) |
| | ACTH (5-10) (H-1195) |
| | (Tyr¹⁵)-ACTH (7-15) (H-1200) |
| | ACTH (7-38) (H-1205) |
| | ACTH (11-24) (H-1210) |
| | ACTH (18-39) (H-1215) (Corticotropin-Like Intermediate Peptide) |
| | ACTH (22-39) (H-2898) |
| | ACTH (34-39) (H-1220) |
| | α-MSH (H-1075) |
| | (Des-acetyl)-α-MSH (H-4390) |
| | ACTH (17-39) (T-1601) (Arg¹⁷-Corticotropin-Like Intermediate Peptide) |
| Adrenomedullins | Adrenomedullin (H-2932) |
| | Adrenomedullin (13-52) (H-4936) |
| | Adrenomedullin (16-31) (H-4064) |
| | Adrenomedullin (22-52) (H-4144) |
| | Adrenomedullin (26-52) (H-4138) |
| | Proadrenomedullin (1-20) (H-4916) |
| | Proadrenomedullin (12-20) (H-3994) |
| Amylins | Amylin (H-7905) |
| | Amylin (1-13) (H-5708) |
| | Amylin (8-37) (H-2742) |
| | Acetyl-Amylin (8-37) (H-2744) |
| | Amylin (20-29) (H-3746) |
| Anti-HIV peptides | Ac-muramyl-Ala-D-Glu-NH₂ (G-1055) |
| | Asn-Ala-Intercellular Adhesion Molecule 1 (1-21) (H-2078) |
| | (Tyr^{5,12},Lys⁷)-Polyphemusin II (H-2694) |
| | Polyphemusin II-Derived Peptide (H-4626) |
| | (Trp¹¹,D-Phe^{15,16})-SDF-1 (7-16) (Dimer) (H-5876) |
| | Tachyplesin I (H-1202) |
| | (Cys(Bzl)⁸⁴)-CD4 (81-92) (H-8085) |
| | (Cys(Bzl)⁸⁴,Glu(OBzl)⁸⁵)-CD4 (81-92) (H-9655) |
| | CDR-H3/C2 (H-1588) |
| | H-His-Cys-Lys-Phe-Trp-Trp-OH (H-3524) |
| | Acetyl-Pepstatin (N-1250) |
| | Ac-Leu-Val-Phe-aldehyde (N-1395) |
| | Ac-Thr-Ile-Nle-(®)-Nle-Gln-Arg-NH₂ (N-1465) |
| | Ac-Thr-Leu-Asn-Phe-OH (H-8540) |
| | Ac-Thr-Val-Ser-Phe-Asn-Phe-OH (H-1956) |
| | H-Arg-Val-Leu-(®)-Phe-Glu-Ala-Nle-NH₂ (N-1270) |
| | H-Ser-Gln-Asn-Phe-(®)-Pro-Ile-Val-Gln-OH (N-1460) |
| Anti-Inflammatory | Anti-Inflammatory Peptide 1 (H-9435) |
| Peptides | Anti-Inflammatory Peptide 2 (H-9440) |
| | Anti-Inflammatory Peptide 3 (H-2806) |
| Anti-Oxidant | L-Anserine (G-4555) |
| Peptides | Carcinine (G-4425) |
| | L-Carnosine (G-1250) |
| | H-Pro-His-Cys-Lys-Arg-Met-OH (H-2458) |
| | H-Pro-Phe-Thr-Arg-Asn-Tyr-Tyr-Val-Arg-Ala-Val-Leu-His-Leu-OH (H-2518) |
| | H-Thr-Arg-Asn-Tyr-Tyr-Val-Arg-Ala-Val-Leu-OH (H-2516) |
| Angiotensins | Angiotensin I (H-1680) |
| | Acetyl-Angiotensin I (H-1015) |
| | Biotinyl-Angiotensin I (H-5736) |
| | (Des-Asp¹)-Angiotensin I (H-1700) |
| | (Val⁵,Asn⁹)-Angiotensin I (H-1695) |
| | Angiotensin I (1-9) (H-5038) |
| | Angiotensin II (H-1705) |
| | (p-Amino-Phe⁶)-Angiotensin II (H-1022) |
| | (Asn¹,Val⁵)-Angiotensin II (H-6010) |
| | (Des-Asp¹,Ile⁸)-Angiotensin II (H-1710) |
| | (3,5-Diiodo-Tyr4)-Angiotensin II (H-2886) |
| | (Sar1)-Angiotensin II (H-1740) |
| | (Sar1,Ala⁸)-Angiotensin II (H-1720) |
| | (Sar1,Gly⁸)-Angiotensin II (H-1725) |
| | (Sar1,Ile⁸)-Angiotensin II (H-1730) |
| | (Sar1,Thr⁸)-Angiotensin II (H-1745) |
| | (Sar1,Tyr(Me)⁴)-Angiotensin II (H-4178) |
| | (Sar1,Val⁵,Ala⁸)-Angiotensin II (H-1232) |
| | (Val⁵)-Angiotensin II (H-1750) |
| | Angiotensin II Antipeptide (H-8160) |
| | Angiotensin II Receptor Ligand (H-9395) |
| | Angiotensin I/II (1-5) (H-2878) |
| | Angiotensin I/II (1-6) (H-2882) |
| | Angiotensin I/II (1-7) (H-1715) |
| | (D-Ala⁷)-Angiotensin I/II (1-7) (H-2888) |
| | Biotinyl-Angiotensin I/II (1-7) (H-4046) |
| | (Sar1)-Angiotensin I/II (1-7) amide (H-2892) |
| | Angiotensin I/II (3-7) (H-6965) |
| | Angiotensin I/II (3-8) (H-8125) |
| | Angiotensin I/II (4-8) (H-2884) |
| | Angiotensin I/II (5-8) (H-3846) |
| | Angiotensin III (H-1755) |
| | (Val⁴)-Angiotensin III (H-1760) |
| Apelins | Apelin-12 (H-5806) |
| | Apelin-13 (H-4566) |
| | (Pyr1)-Apelin-13 (H-4568) |
| | (Tyr0)-Apelin-13 (H-4894) |
| | Apelin-36 (H-4896) |
| BAM Peptides | BAM-12P (H-2125) |
| | BAM-12P (7-12) (H-5365) |
| | BAM-22P (H-2130) |
| | BAM-3200 (H-4500) |
| Basic Fibroblast | H-Ala-Pro-Ser-Gly-His-Tyr-Lys-Gly-OH (H-1948) |
| Growth Factor | FGF basic (119-126) (H-1952) |
| (FGF) Inhibitory | H-Met-Trp-Tyr-Arg-Pro-Asp-Leu-Asp-Glu-Arg-Lys-Gln-Gln-Lys-Arg-Glu-OH |
| Peptides | (H-2176) |
| Bombesins | Bombesin (H-2155) |
| | (Leu¹³-(®)-Leu¹⁴)-Bombesin (H-7075) |
| | (Lys³)-Bombesin (H-2160) |
| | (D-Phe¹²)-Bombesin (H-3038 |
| | (D-Phe¹²,Leu¹⁴)-Bombesin (H-7070) |
| | (Tyr⁴)-Bombesin (H-2165) |
| | (Tyr⁴,D-Phe¹²)-Bombesin (H-9065) |
| | (D-Cys⁶,Asn⁷,D-Ala¹¹,Cys¹⁴)-Bombesin (6-14) (H-8465) |
| | (D-Phe⁶,Leu¹³-(®)-p-chloro-Phe¹⁴)-Bombesin (6-14) (H-3028) |
| | (D-Phe⁶,Leu-NHEt¹³,des-Met¹⁴)-Bombesin (6-14) (H-3042) |
| | Bombesin (8-14) (H-2170) |
| | Cyclo(-D-Phe-His-Trp-Ala-Val-Gly-His-Leu-Leu) (H-8470) |
| Bradykinins | Bradykinin (H-1970) |
| | (1-Adamantaneacetyl-D-Arg⁰,Hyp³,β-(2-thienyl)-Ala^{5,8},D-Phe⁷)-Bradykinin (H- |
| | 1114) |
| | (1-Adamantanecarbonyl-D-Arg⁰,Hyp³,β-(2-thienyl)-Ala^{5,8},D-Phe⁷)-Bradykinin |
| | (H-1116) |
| | (D-Arg⁰,Hyp^{2,3},D-Phe⁷)-Bradykinin (H-9090) |
| | (D-Arg⁰,Hyp³,D-Phe⁷)-Bradykinin (H-6385) |
| | (D-Arg⁰,Hyp³,D-Phe⁷,Leu⁸)-Bradykinin (H-1652) |
| | D-Arg⁰,Hyp³,β-(2-thienyl)-Ala^{5,8},D-Phe⁷)-Bradykinin (H-6560) |
| | (p-Chloro-Phe^{5,8})-Bradykinin (H-1940) |
| | (3,4-Dehydro-Pro^{2,3})-Bradykinin (H-3132) |
| | (3,4-Dehydro-Pro^{2,3},des-Arg⁹)-Bradykinin (H-3124) |
| | (Des-Arg¹)-Bradykinin (H-2200) |
| | (Des-Arg⁹)-Bradykinin (H-1965) |
| | (Des-Arg⁹,Leu⁸)-Bradykinin (H-1960) |
| | (Hyp³)-Bradykinin (H-5465) |
| | (N-Me-D-Phe⁷)-Bradykinin (H-5094) |
| | (D-Phe⁷)-Bradykinin (H-9085) |
| | (β-(2-Thienyl)-Ala^{5,8},D-Phe⁷)-Bradykinin (H-9080) |
| | (Thr⁶)-Bradykinin (H-6325) |
| | (Tyr⁸)-Bradykinin (H-1975) |
| | Lys-Bradykinin (H-2180) (also known as kallidin) |
| | Lys-Bradykinin-Ser-Val-Gln-Val-Ser (H-59250 |
| | Lys-(Ala³)-Bradykinin (H-9535) |
| | Lys-(Des-Arg⁹)-Bradykinin (H-3122) |
| | Lys-(Des-Arg⁹,Leu⁸)-Bradykinin (H-2582) |
| | Lys-(Hyp³)-Bradykinin (H-9075) |
| | Lys-(Tyr⁸)-Bradykinin (H-4378) |
| | Lys-Lys-(Hyp³,β-(2-thienyl)-Ala^{5,8},D-Phe⁷)-Bradykinin (H-9070) |
| | Mca-(Ala⁷,Lys(Dnp)⁹)-Bradykinin (M-2405) |
| | Met-Lys-Bradykinin (H-2190) |
| | Tyr-Bradykinin (H-2195) |
| | Bradykinin-Like Neuropeptide (3-11) (H-1656) |
| | H-Met-Lys-Arg-Ser-Arg-Gly-Pro-Ser-Pro-Arg-Arg-OH (H-1654) |
| Bradykinin- | A-VI-5 (H-2220) |
| Potentiating | Angiotensin I-Converting Enzyme Substrate (H-9050) |
| Peptides (BPP) | BPP 5a (H-2225) |
| | BPP 9a (H-2215) |
| | Bradykinin Potentiator B (H-2205) |
| | Bradykinin Potentiator C (H-2210) |
| | H-Pro-Thr-His-Ile-Lys-Trp-Gly-Asp-OH (N-1450) |
| | H-Val-Trp-OH (N-1170) |
| C3a and C3d | (Trp⁶³,Trp⁶⁴)-C3a (63-77) (H-1264) |
| Peptides | (Tyr⁶⁹,Ala^{71,72},Lys⁷⁴)-C3a (69-77) (H-1432) |
| | C3a (70-77) (H-1645) |
| | (β-Ala⁷⁰)-C3a (70-77) (H-1650) |
| | (Fmoc-Glu⁷⁰,Ala^{71,72},Lys⁷⁴)-C3a (70-77) (B-2280) |
| | C3a (72-77) (H-2235) |
| | C3d Peptide P16 (H-1374) |
| C5a-Related | (Tyr⁶⁵,Phe⁶⁷)-C5a (65-74) (H-3462) |
| Peptides | C5a Inhibitory Sequence (H-8135) |
| Caerulein | Caerulein (H-3220) |
| | Caerulein (desulfated) (H-2245) |
| | Boc-(Asp(OBzl)16)-Gastrin I (13-17) (A-4310) |
| Calcitonin and | Calcitonin (H-2250) |
| Calcitonin | Calcitonin C-Terminal Flanking Peptide (H-2050) (also known as C- |
| Precursors | Procalcitonin) |
| | Calcitonin N-Terminal Flanking Peptide (H-3076) (also known as N- |
| | Procalcitonin) |
| Calcitonin Gene- | α-CGRP (H-1470) |
| Related Peptides | (Cys(Acm)^{2,7})-α-CGRP (H-5766) |
| (CGRP) | (Cys(Et)^{2,7})-α-CGRP (H-5784) |
| | Tyr-α-CGRP (H-3354) |
| | α-CGRP (8-37) (H-9895) |
| | Acetyl-α-CGRP (19-37) (H-8890) |
| | α-CGRP (19-37) (H-8885) |
| | α-CGRP (23-37) (H-8895) |
| | β-CGRP (H-6730) |
| Calpain Inhibitors | Acetyl-Calpastatin (184-210) (H-4076) |
| | Calpain Inhibitor I (N-1320) |
| | Calpain Inhibitor II (N-1315) |
| | Calpain Inhibitor III (N-1535) |
| | Calpain Inhibitor IV (N-1635) |
| α-Casein | α-Casein (90-95) (H-2000) |
| Exorphins | α-Casein (90-96) (H-2005) |
| β-Casomorphins | β-Casomorphin (H-2275) |
| | β-Casomorphin (1-2) (G-3625) |
| | β-Casomorphin (1-2) amide (G-3457) |
| | β-Casomorphin (1-3) (H-2375) |
| | β-Casomorphin (1-3) amide (H-2380) |
| | (D-Ala²)-β-Casomorphin (1-3) amide (H-2385) |
| | (D-Ala²,Hyp⁴,Tyr⁵)-β-Casomorphin (1-5) amide (H-2310) |
| Cathepsin G | Cathepsin G (77-83) (H-1266) |
| Peptides | Cathepsin G (77-83) amide (H-8240) |
| Cecropins | Cecropin A (1-8)-Melittin (1-18) amide (H-4314) |
| | Cecropin B (H-3096) |
| | Cecropin P1 (H-5718) |
| | Cecropin A (H-3094) |
| Ceratotoxins | Ceratotoxin A (H-1616) |
| | Ceratotoxin B (H-1618) |
| Cerebellins | Cerebellin (H-5530) |
| | (Des-Ser¹)-Cerebellin (H-5535) |
| Cholecystokinin- | Cholecystokinin Octapeptide (H-2080) (Sincalide) |
| Pancreozymin | (Tyr⁹)-Cholecystokinin Octapeptide (H-9770) |
| Peptides | Cholecystokinin Octapeptide (1-4) (H-2060) |
| | Acetyl-Cholecystokinin Octapeptide (2-8) (H-1120) |
| | Boc-Cholecystokinin Octapeptide (3-8) (A-2650) |
| | Cholecystokinin Octapeptide (3-8) (H-2425) |
| | Cholecystokinin-33 (H-5476) |
| | (Thr²⁸,Nle³¹)-Cholecystokinin-33 (25-33) (H-1825) |
| | Boc-(Asp(OBzl)¹⁶)-Gastrin I (13-17) (A-4310) |
| | Gastrin Tetrapeptide (H-3110) |
| Chorionic | Chorionic Gonadotropin-b (109-119) amide (H-1378) |
| Gonadotropin | Chorionic Gonadotropin-b (109-145) (H-6740) |
| (hCG) Peptides | |
| CKS-17 | CKS-17 (H-7600) |
| | CKS-17 (7-12) (H-1442) |
| Cocaine and | CART (55-102) (H-4444) |
| Amphetamine | CART (61-102) (H-4448) |
| Regulated | CART (62-76) (H-5098) |
| Transcript (CART) | |
| Peptides | |
| Conantokin G | Conantokin G (H-9960) |
| peptides | (Glu^{3,4,7,10,14})-Conantokin G (H-1236) |
| | (Tyr⁰)-Conantokin G (H-8130) |
| | Conantokin G (H-2156) |
| Corticotropin- | Astressin (H-3422) |
| Releasing Factor | CRF (H-2440) |
| (CRF) and | Tyr-CRF (H-2455) |
| Analogs | CRF (6-33) (H-3456) |
| | (D-Phe¹²,Nle^{21,38})-CRF (12-41) (H-5482) |
| | (D-Phe¹²,Nle^{21,38},α-Me-Leu37)-CRF (12-41) (H-3266) |
| | α-Helical CRF (9-41) (H-2040) |
| | α-Helical CRF (12-41) (H-3268) |
| C-Reactive Protein | C-Reactive Protein (CRP) (77-82) (H-1436) |
| (CRP) Sequences | C-Reactive Protein (CRP) (174-185) (H-1344) |
| | C-Reactive Protein (CRP) (201-206) (H-1438) |
| Defensins | Defensin HNP-1 (H-9855) |
| | Defensin HNP-2 (H-9005) |
| | rec β-Defensin 1 (H-5584) |
| | rec β-Defensin 2 (H-5586) |
| Delta-Sleep | Delta-Sleep Inducing Peptide (H-2540) |
| Inducing Peptides | (Asn⁵)-Delta-Sleep Inducing Peptide (H-2555) |
| (DSIP) | (β-Asp⁵)-Delta-Sleep Inducing Peptide (H-2545) |
| | (Tyr¹)-Delta-Sleep Inducing Peptide (H-2560) |
| Deltorphins, and | Deltorphin (H-8090) |
| Dermorphins | (Met²)-Deltorphin (H-9355) |
| | Deltorphin I (H-8055) |
| | Deltorphin II (H-8060) |
| | Dermorphin (H-2565) |
| | (Ser(Ac)⁷)-Dermorphin (H-6595) |
| | (D-Arg²,Sar⁴)-Dermorphin (1-4) (H-3568) |
| | (D-Arg²)-Dermorphin (1-4) amide (H-6755) |
| | (D-Arg²,Lys⁴)-Dermorphin (1-4) amide (H-8865) |
| | (Phe⁴)-Dermorphin (1-4) amide (H-8870) |
| Eglin c peptides | Eglin c (H-7770) |
| | Eglin c (41-49) (H-2474) |
| | Eglin c (42-45)-methyl ester (H-1184) |
| | Eglin c (60-63)-methyl ester (H-8150) |
| Endomorphins | Endomorphin-1 (H-4002) |
| | Endomorphin-2 (H-4004) |
| Endorphins | α-Endorphin (H-2695) |
| | β-Endorphin (H-2700) |
| | Acetyl-β-Endorphin (H-1115) |
| | β-Endorphin (6-31) (H-4024) |
| | β-Endorphin (18-31) (H-5686) |
| | β-Endorphin (27-31) (H-5170) |
| | β-Endorphin (30-31) (G-2080) |
| | δ-Endorphin (H-2710) |
| | γ-Endorphin (H-2725) |
| Endothelin | Azepane-1-carbonyl-Leu-D-Trp(For)-D-Trp-OH (H-4914) |
| Antagonists | Cyclo(-D-Asp-Pro-D-Ile-Leu-D-Trp) (H-3008) |
| | Cyclo(-D-Glu-Ala-D-allo-Ile-Leu-D-Trp) (H-8405) |
| | Cyclo(-D-Ser-Pro-D-Val-Leu-D-Trp) (H-3064) |
| | Cyclo(-D-Trp-D-Asp-Pro-D-Val-Leu) (H-1252) |
| | N-cis-2,6-Dimethylpiperidinocarbonyl-b-tBu-Ala-D-Trp(1-methoxycarbonyl)-D- |
| | Nle-OH (H-2492) |
| | Endothelin-1 (11-21) (H-1658) |
| | Acetyl-(D-Trp¹⁶)-Endothelin-1 (16-21) (H-8850) |
| Enkephalins and | Dynorphin A (1-7) (H-2660) |
| Proenkephalins | (Phe⁷)-Dynorphin A (1-7) (H-5150) |
| | (Phe⁷)-Dynorphin A (1-7) amide (H-5155) |
| | Dynorphin A (1-6) (H-2665) |
| | Dynorphin A (1-13) (H-2625) |
| | Gluten Exorphin B5 (H-1666) |
| | Leu-Enkephalin (H-2740) |
| | Leu-Enkephalin (sulfated) (H-2760) |
| | (Ala²)-Leu-Enkephalin (H-1276) |
| | (D-Ala²)-Leu-Enkephalin (H-2750) |
| | (Des-Tyr¹)-Leu-Enkephalin (N-1175) |
| | (3,5-Dibromo-Tyr¹)-Leu-Enkephalin (H-2575) |
| | Boc-Leu-Enkephalin (A-2440) |
| | Leu-Enkephalin amide (H-2745) |
| | (D-Ala²)-Leu-Enkephalin amide (H-2755) |
| | (D-Ala²)-Leu-Enkephalin-Arg (H-3276) |
| | (Boc-Tyr¹,D-Ala²)-Leu-Enkephalin-Lys (A-2435) |
| | Leu-Enkephalin-Lys (H-2765) |
| | (D-Cys(tBu)²,Thr(tBu)⁶)-Leu-Enkephalin-Thr (H-8170) |
| | (3,5-Diiodo-Tyr¹,D-Thr²)-Leu-Enkephalin-Thr (H-2615) |
| | (D-Ser²)-Leu-Enkephalin-Thr (H-2770) |
| | (D-Thr²)-Leu-Enkephalin-Thr (H-2775) |
| | Met-Enkephalin (H-2785) |
| | (Des-Tyr¹)-Met-Enkephalin (N-1180) |
| | (Gly⁰)-Met-Enkephalin (H-2850) |
| | (Met(O)⁵)-Enkephalin (H-5160) |
| | Boc-Met-Enkephalin (A-2445) |
| | Boc-Met-Enkephalin-t-butyl ester (A-2815) |
| | (3,5-Diiodo-Tyr¹,D-Ala²)-Met-Enkephalin amide (H-2600) |
| | Met-Enkephalin-Arg (H-2805) |
| | (Met(O)⁵)-Enkephalin-Arg (H-2810) |
| | Met-Enkephalin-Arg-Arg (H-2815) |
| | Met-Enkephalin-Arg-Gly-Leu (H-2820) |
| | Met-Enkephalin-Arg-Lys (H-2825) |
| | Met-Enkephalin-Arg-Phe (H-2830) |
| | Met-Enkephalin-Arg-Phe amide (H-2835) |
| | Met-Enkephalin-Lys (H-1340) |
| | Met-Enkephalin-Lys-Arg (H-2840) |
| | Met-Enkephalin-Lys-Lys (H-2845) |
| | H-Tyr-D-Ala-Gly-Phe-Met-NH₂ (H-2795) |
| | (D-Ala²,D-Leu⁵)-Enkephalin (H-2860) |
| | (D-Ala²,D-Leu⁵)-Enkephalin amide (H-2865) |
| | (D-Ala²,D-Leu⁵)-Enkephalin (H-2860) |
| | (D-Ala²,N-Me-Phe⁴,glycinol⁵)-Enkephalin (H-2535) |
| | (D-Ala²,N-Me-Phe⁴,methionin(O)-ol⁵)-Enkephalin (H-8270) |
| | (3,5-Diiodo-Tyr¹,D-Ala²,N-Me-Phe⁴,glycinol⁵)-Enkephalin (H-2595) |
| | (Guanyl-Tyr¹,D-Ala²,N-Me-Phe⁴,methionin(O)-ol⁵)-Enkephalin (H-8275) |
| | (m-Iodo-Tyr¹,D-Ala²,N-Me-Phe⁴,methionin(O)-ol⁵)-Enkephalin (H-3656) |
| | (D-Pen²,p-chloro-Phe⁴,D-Pen⁵)-Enkephalin (H-8875) |
| | (D-Pen²,Pen⁵)-Enkephalin (H-2900) |
| | (D-Pen²,D-Pen⁵)-Enkephalin (H-2905) |
| | (D-Cys(tBu)²,Thr(tBu)⁶)-Leu-Enkephalin-Thr (H-8170) |
| | (3,5-Diiodo-Tyr¹,D-Thr²)-Leu-Enkephalin-Thr (H-2615) |
| | (D-Ser²)-Leu-Enkephalin-Thr (H-2770) |
| | (D-Thr²)-Leu-Enkephalin-Thr (H-2775) |
| Farnesyltransferase | H-Cys-4-Abz-Met-OH (H-3548) |
| Inhibitors | H-Cys-Val-2-Nal-Met-OH (H-3552) |
| | H-Cys-(®)-Val-(®)-Phe-Met-OH (N-1390) |
| | H-D-Trp-D-Met-p-chloro-D-Phe-Gla-NH₂ (N-1665) |
| FIV Peptide | Ac-1-Nal-Abu-Phe-(®)-Abu-Abu-1-Nal-NH₂ (N-1705) |
| FMRFamide | ACEP-1 (H-1646) |
| Peptides | Ac-Phe-Nle-Arg-Phe-NH₂ (H-1055) |
| | AF-1 (H-3338) |
| | AF-2 (H-1642) |
| | H-Asn-Arg-Asn-Phe-Leu-Arg-Phe-NH₂ (H-1364) |
| | H-Asp-Arg-Asn-Phe-Leu-Arg-Phe-NH₂ (H-1362) |
| | H-Leu-Ser-Ser-Phe-Val-Arg-Ile-NH₂ (H-1644) |
| | H-Met-Arg-Phe-OH (H-2965) |
| | Met-Enkephalin-Arg-Phe (H-2830) |
| | Met-Enkephalin-Arg-Phe amide (H-2835) |
| | H-Nle-Arg-Phe-NH₂ (H-2970) |
| | Orphan GPCR SP9155 Agonist P518 (H-5984) |
| | H-Phe-Leu-Arg-Phe-NH₂ (H-2985) |
| | H-Phe-Met-Arg-Phe-NH₂ (H-2975) |
| | H-D-Phe-Met-Arg-Phe-NH₂ (H-3346) |
| | H-Phe-D-Met-Arg-Phe-NH₂ (H-3344) |
| | H-Phe-Met-Arg-D-Phe-NH₂ (H-3342) |
| | H-Phe-Met-D-Arg-Phe-NH₂ (H-7185) |
| | H-Pro-Asp-Val-Asp-His-Val-Phe-Leu-Arg-Phe-NH₂ (H-8040) |
| | Pyr-Asp-Pro-Phe-Leu-Arg-Phe-NH₂ (H-9260) |
| | SCPA (H-6925) |
| | SCPB (H-3005) |
| | H-Thr-Asn-Arg-Asn-Phe-Leu-Arg-Phe-NH₂ (H-9265) |
| | H-Trp-Nle-Arg-Phe-NH₂ (H-3000) |
| | H-Tyr-Phe-Met-Arg-Phe-NH₂ (H-2980) |
| Galanins and | Galanin (H-8230) |
| Galanin Message | (Abz-Gly¹)-Galanin (1-10)-Lys(retro-m-nitro-Tyr-H) amide (M-2365) |
| Associated | Galanin (1-13)-Bradykinin (2-9) amide (H-1346) |
| Peptides (GMAP) | Galanin (1-13)-Mastoparan (H-4188) |
| | Galanin (1-13)-Neuropeptide Y (25-36) amide (H-3374) |
| | Galanin (1-13)-Pro-Pro-(Ala-Leu-)2Ala amide (H-2576) |
| | Galanin (1-13)-Spantide I (H-2578) |
| | Galanin (1-13)-Substance P (5-11) amide (H-1312) |
| | (Ala⁶,D-Trp⁸,L-alaninol¹⁵)-Galanin (1-15) (H-4066) |
| | (D-Thr⁶,D-Trp^{8,9},L-alaninol¹⁵)-Galanin (1-15) (H-1576) |
| | Galanin (1-19) (H-5754) |
| | (D-Trp²)-Galanin (1-29) (H-4122) |
| | Galanin Message Associated Peptide (1-41) amide (H-6780) |
| | Galanin Message Associated Peptide (1-41) amide (H-7615) |
| | Galanin Message Associated Peptide (16-41) amide (H-9725) |
| | Galanin Message Associated Peptide (25-41) amide (H-9520) |
| | Galanin Message Associated Peptide (44-59) amide (H-7715) |
| | Galnon (B-3645) |
| Gastrins | Big Gastrin I (H-7320) |
| | Gastrin I (H-3085) |
| | Gastrin I (sulfated) (H-9170) |
| | (Leu15)-Gastrin I (H-3090) |
| | Gastrin I (1-14) (H-3095) |
| | Boc-(Asp(OBzl)16)-Gastrin I (13-17) (A-4310) |
| | Gastrin I (rat) (H-9165) |
| | Gastrin Tetrapeptide (H-3110) |
| | Minigastrin I (H-3105) |
| | Pentagastrin (A-1130) |
| Gastrin Releasing | GRP (H-6785) |
| Peptides (GRP) | GRP (14-27) (H-3115) |
| | GRP (18-27) (H-3120) |
| | (Deamino-Phe19,D-Ala24,D-Pro26-(®)-Phe27)-GRP (19-27) (H-2756) |
| | Acetyl-GRP (20-26) (H-6705) |
| | Acetyl-GRP (20-27) (H-1040) |
| Ghrelins | Ghrelin (H-4864) |
| | (Des-octanoyl)-Ghrelin (H-5946) |
| Glucagons and | GLP-1 (1-36) amide (H-6025) |
| Glucagon-Like | GLP-1 (1-37) (H-5552) |
| Peptides | GLP-1 (7-36) amide (H-6795) |
| | (Ser⁸)-GLP-1 (7-36) amide (H-4592) |
| | GLP-1 (7-37) (H-5102) |
| | GLP-2 (H-5662) |
| | GLP-2-Arg (H-4766) |
| | Glucagon (1-29) (H-6790) |
| | (Des-His1,Glu⁹)-Glucagon (1-29) amide (H-2754) |
| | Glucagon (19-29) (H-2758) |
| Gluten Exorphins | Gluten Exorphin A5 (H-1668) |
| | Gluten Exorphin B5 (H-1666) |
| | Gluten Exorphin C (H-1412) |
| GM-CSF | GM-CSF (17-31) (H-3436) |
| Inhibitory | Cys-GM-CSF (17-31) (H-3474) |
| Peptides | GM-CSF (96-112) (H-3442) |
| Growth Hormone- | GRF (1-29) amide (H-3705) |
| Releasing Factors | GRF (1-29) amide (H-3710) |
| (GRF) and | Acetyl-(Tyr¹,D-Arg²)-GRF (1-29) amide (H-5560) |
| Peptides (GHRP) | Acetyl-(Tyr¹,D-Phe²)-GRF (1-29) amide (H-5565) |
| | (D-Ala²)-GRF (1-29) amide (H-3715) |
| | (Nle²⁷)-GRF (1-29) amide (H-6030) |
| | (Phenylac-Tyr¹,D-Arg²,p-chloro-Phe⁶,Arg⁹,Abu¹⁵,Nle²⁷,D-Arg²⁸,Homoarg²⁹)- |
| | GRF (1-29) amide (H-4884) |
| | (Phenylac-Tyr¹,D-Arg²,p-chloro-Phe⁶,Homoarg⁹,Tyr(Me)¹⁰,Abu¹⁵,Nle²⁷,D- |
| | Arg²⁸,Homoarg²⁹)-GRF (1-29) amide (H-4886) |
| | GRF (1-40) (H-3685) |
| | GRF (free acid) (H-4686) |
| | GRF (H-3695) |
| | GRF (H-3700) |
| | (β-Asp³)-GRF (H-4688) |
| | (Met(O)²⁷)-GRF (H-4692) |
| | GHRP-2 (H-5558) |
| | (Des-Ala³)-GHRP-2 (H-2528) |
| | (D-Lys³)-GHRP-6 (H-3108) |
| | (D-Trp⁷,Ala⁸,D-Phe¹⁰)-a-MSH (6-11) amide (H-9990) |
| Helodermins | Helodermin (H-5696) |
| | (Glu^{8,9})-Helodermin (H-5062) |
| Hirudins | Acetyl-Hirudin (53-65) (sulfated) (H-8190) |
| | Hirudin (54-65) (desulfated) (H-7420) |
| | Hirudin (54-65) (sulfated) (H-7425) |
| | Acetyl-Hirudin (54-65) (sulfated) (H-7415) |
| | Acetyl-Hirudin (55-65) (desulfated) (H-7430) |
| | Hirudin (55-65) (sulfated) (H-7445) |
| | Acetyl-Hirudin (55-65) (sulfated) (H-7435) |
| | Succinyl-(Pro⁵⁸,D-Glu⁶⁵)-Hirudin (56-65) (sulfated) (H-8145) |
| Hylambatins | Hylambatin (H-9320) |
| | Entero-Hylambatin (H-9325) |
| Insulin-like | H-Asn-Pro-Glu-Tyr(PO3H2)-OH (H-2706) |
| growth factors | rec IGF-I (H-5555) |
| (IGF) | (Des-Met⁰)-rec IGF-I (H-3102) |
| | IGF-I Analog (H-1356) |
| | IGF-I (1-3) (H-2468) |
| | IGF-I (24-41) (H-3098) |
| | IGF-I (30-41) (H-7460) |
| | rec IGF-II (1-67) (H-7020) |
| | IGF-II (33-40) (H-7250) |
| Interleukins, | IL-1α (223-250) (H-8290) |
| Fragments and | IL-1β (163-171) (H-7010) |
| Related Peptides | IL-1β (178-207) (H-8300) |
| | (D-Pro194)-IL-1b (193-195) (H-7230) |
| | IL-1β (208-240) (H-8285) |
| | IL-2 (H-7365) |
| | IL-3 (H-7730) |
| | IL-4 (H-9630) |
| | IL-6 (H-7735) |
| | IL-6 (88-121) (H-1398) |
| | IL-7 (H-9635) |
| | IL-8 (−5 to +5) (H-3564) |
| | Endothelial IL-8 (H-3742) |
| | Monocyte IL-8 (H-9625) |
| | IL-8 Inhibitor (H-2268) |
| | IL-10 (H-8805) |
| | IL-11 (H-1702) |
| Kinetensins | Kinetensin (H-9350) |
| | (Des-Leu⁹)-Kinetensin (H-1358) |
| Kyotorphins | Kyotorphin (G-2450) |
| | (D-Arg²)-Kyotorphin (G-2455) |
| | Neo-Kyotorphin (H-3845) |
| Laminins | H-Arg-Asn-Ile-Ala-Glu-Ile-Ile-Lys-Asp-Ile-OH (H-1016) |
| | H-Arg-Gly-Asp-OH (H-1830) |
| | H-Cys-Asp-Pro-Gly-Tyr-Ile-Gly-Ser-Arg-OH (H-2798) |
| | H-Cys-Asp-Pro-Gly-Tyr-Ile-Gly-Ser-Arg-NH₂ (H-1224) |
| | H-Leu-Gln-Val-Gln-Leu-Ser-Ile-Arg-OH (H-4588) |
| | H-Ser-Ile-Lys-Val-Ala-Val-OH (H-2684) |
| | H-Tyr-Ile-Gly-Ser-Arg-OH (H-6825) |
| | H-Tyr-Ile-Gly-Ser-Arg-NH₂ (H-2802) |
| Leptins | Leptin (22-56) (H-3424) |
| | Tyr-Leptin (26-39) (H-3494) |
| | Leptin (93-105) (H-3426) |
| | Leptin (126-140) (H-3492) |
| | Leptin (138-167) (H-3428) |
| | Leptin (150-167) (H-3432) |
| Leucokinins | Leucokinin I (H-6835) |
| | Leucokinin II (H-6830) |
| | Leucokinin III (H-9240) |
| | Leucokinin IV (H-9245) |
| Leupeptins | Leupeptin (N-1000) |
| | Ac-Leu-Val-Lys-aldehyde (N-1380) |
| | Calpain Inhibitor I (N-1320) |
| | Calpain Inhibitor II (N-1315) |
| Luteinizing | antide (H-9215) |
| hormone-releasing | LHRH (H-4005) (also known as gonadorelin) |
| Hormone Peptides | Acetyl-(3,4-dehydro-Pro¹,4-fluoro-D-Phe²,D-Trp^{3,6})-LHRH (H-4050) |
| | Acetyl-(D-Trp¹,4-chloro-D-Phe²,D-Trp³,D-Arg⁶,D-Ala¹⁰)-LHRH (H-5575) |
| | (D-Ala⁶)-LHRH (H-4020) |
| | (Des-Gly¹⁰,D-Ala⁶,Pro-NHEt⁹)-LHRH (H-4070) |
| | (Des-Gly¹⁰,D-His²,D-His(Bzl)⁶,Pro-NHEt⁹)-LHRH (H-4652) |
| | (Des-Gly¹⁰,D-His²,D-Leu⁶,Pro-NHEt⁹)-LHRH (H-4316) |
| | (Des-Gly¹⁰,D-His²,D-Trp⁶,Pro-NHEt⁹)-LHRH (H-4986) |
| | (Des-Gly¹⁰,D-His(Bzl)⁶,D-Leu⁷,Pro-NHEt⁹)-LHRH (H-4658) |
| | (Des-Gly¹⁰,His(Bzl)⁶,Pro-NHEt⁹)-LHRH (H-4656) |
| | (Des-Gly¹⁰,D-His(Bzl)⁶,Pro-NHEt⁹)-LHRH (H-9210) (also known as histrelin) |
| | (Des-Gly¹⁰,D-Leu⁶,D-Leu⁷,Pro-NHEt⁹)-LHRH (H-4636) |
| | (Des-Gly¹⁰,D-Leu⁶,Pro-NHEt⁹)-LHRH (H-4060) (also known as leuprolide) |
| | (Des-Gly¹⁰,D-Ser⁴,D-His(Bzl)⁶,Pro-NHEt⁹)-LHRH (H-4704) |
| | (Des-Gly¹⁰,D-Ser(tBu)⁶,Pro-NHEt⁹)-LHRH (H-4224) (also known as |
| | buserelin) |
| | (Des-Gly¹⁰,D-Ser⁴,D-Trp⁶,Pro-NHEt⁹)-LHRH (H-4988) |
| | (Des-Gly¹⁰,D-Trp⁶,D-Leu⁷,Pro-NHEt⁹)-LHRH (H-4994) |
| | (Des-Gly¹⁰,D-Trp6,Pro-NHEt⁹)-LHRH (H-4065) (also known as deslorelin) |
| | (Des-Gly¹⁰,D-Tyr5,D-His(Bzl)⁶,Pro-NHEt⁹)-LHRH (H-4654) |
| | (Des-Gly¹⁰,D-Tyr5,D-Leu⁶,Pro-NHEt⁹)-LHRH (H-4638) |
| | (Des-Gly¹⁰,D-Tyr5,D-Trp⁶,Pro-NHEt⁹)-LHRH (H-4992) |
| | (Des-Gly¹⁰,Pro-NHEt⁹)-LHRH (H-4055) (also known as fertirelin) |
| | (Des-Pyr¹)-LHRH (H-9200) |
| | (3,5-Diiodo-Tyr⁵)-LHRH (H-1375) |
| | (His(1-Me)²)-LHRH (H-5405) |
| | (His(3-Me)²)-LHRH (H-4492) |
| | (D-His²,D-Ser(tBu)⁶,Azagly¹⁰)-LHRH (H-5796) |
| | (D-His²,D-Trp⁶)-LHRH (H-4642) |
| | (D-Leu⁷)-LHRH (H-5958) |
| | (D-Lys⁶)-LHRH (H-4025) |
| | (D-Phe²⁶,Pro³)-LHRH (H-4045) |
| | (D-Pyr¹,D-Phe²,D-Trp^{3,6})-LHRH (H-4040) |
| | (D-Ser⁴)-LHRH (H-4706) |
| | (D-Ser⁴,D-Ser(tBu)⁶,Azagly¹⁰)-LHRH (H-5654) |
| | (D-Ser⁴,D-Trp⁶)-LHRH (H-4644) |
| | (D-Ser(tBu)⁶,Azagly¹⁰)-LHRH (H-6395) (also known as goserelin) |
| | (D-Ser(tBu)⁶,D-Leu⁷,Azagly¹⁰)-LHRH (H-5418) |
| | (Trp⁶)-LHRH (H-4578) |
| | (D-Trp⁶)-LHRH (H-4075) (also known as triptorelin) |
| | (D-Trp⁶)-LHRH-Leu-Arg-Pro-Gly amide (H-4582) |
| | (D-Trp⁶,D-Leu⁷)-LHRH (H-4648) |
| | (D-Tyr⁵,D-Ser(tBu)⁶,Azagly¹⁰)-LHRH (H-5734) |
| | (D-Tyr⁵,D-Trp⁶)-LHRH (H-4646) |
| | LHRH (1-6) amide (H-4494) |
| | (D-Trp⁶)-LHRH (1-6) amide (H-4574) |
| | (D-His(Bzl)⁶)-LHRH (1-7) (H-4804) |
| | LHRH (1-6) (H-5632) |
| | (D-His(Bzl)⁶,Pro-NHEt⁹)-LHRH (2-9) (H-4806) |
| | Formyl-LHRH (2-10) (H-1380) |
| | Formyl-(D-Trp⁶)-LHRH (2-10) (H-4576) |
| | (D-His(Bzl)⁶,Pro-NHEt⁹)-LHRH (3-9) (H-4808) |
| | LHRH (3-10) (H-5735) |
| | (D-His(Bzl)⁶,Pro-NHEt⁹)-LHRH (4-9) (H-4802) |
| | (D-Leu⁶,Pro-NHEt⁹)-LHRH (4-9) (H-4008) |
| | LHRH (4-10) (H-3728) |
| | Nafarelin (H-6095) |
| Mastoparans | Mastoparan (H-3810) |
| | Mastoparan 7 (H-3002) |
| | Mastoparan 17 (H-3004) |
| | Mastoparan X (H-9445) |
| | Polistes Mastoparan (H-9450) |
| Melanin- | (D-Bpa¹³,Tyr¹⁹)-MCH (H-2222) |
| Concentrating | (Phe¹³,Tyr¹⁹)-MCH (H-2218) |
| Hormones (MCH) | MCH (H-1482) |
| Melanocyte- | Melanocyte-Stimulating Hormone-Release Inhibiting Factor (H-4305) |
| Stimulating | (melanostatin) |
| Hormone-Release | (D-Leu²)-Melanocyte-Stimulating Hormone-Release Inhibiting Factor (H-9225) |
| Inhibiting Factors | (Tyr⁰)-Melanocyte-Stimulating Hormone-Release Inhibiting Factor (H-5120) |
| (MIF-I) | (Tyr⁰,Trp²)-Melanocyte-Stimulating Hormone-Release Inhibiting Factor (H- |
| | 8825) |
| Melanotropin- | Melanotropin-Potentiating Factor (H-4170) (also known as β-Lipotropin (88-91)) |
| Potentiating | Acetyl-(D-Lys²,Sar³)-Melanotropin-Potentiating Factor (H-2512) |
| Factors (MPF) | |
| Motilins | Motilin (H-4385) |
| | (Nle¹³,Glu¹⁴)-Motilin (H-4376) |
| Melanin- | MSH-Tetrapeptide (H-3750) |
| Stimulating | (D-Lys³)-GHRP-6 (H-3108) |
| Hormone (MSH) | α-MSH (H-1075) |
| Peptides | (Des-acetyl)-α-MSH (H-4390) |
| | (Diacetyl)-α-MSH (H-7080) |
| | (Nle⁴)-α-MSH (H-1095) |
| | (Nle⁴,D-Phe⁷)-α-MSH (H-1100) |
| | Acetyl-(Cys³,Nle⁴,Arg⁵,D-2-Nal⁷,Cys¹¹)-α-MSH (3-11) amide (H-4598) |
| | (Deamino-Cys³,Nle⁴,Arg⁵,D-2-Nal⁷,Cys¹¹)-α-MSH (3-11) amide (H-4944) |
| | Acetyl-(Nle⁴,Asp⁵,D-2-Nal⁷,Lys¹⁰)-cyclo-α-MSH (4-10) amide (H-3952) |
| | Acetyl-(Nle⁴,Asp⁵,D-Phe⁷,Lys¹⁰)-cyclo-α-MSH (4-10) amide (H-3902) |
| | Acetyl-(Nle⁴,Asp⁵,D-Tyr⁷,Lys¹⁰)-cyclo-α-MSH (4-10) amide (H-5466) |
| | Acetyl-(Nle⁴,Gln⁵,D-Phe⁷,D-Trp⁹)-α-MSH (4-10) amide (H-3594) |
| | Acetyl-(Cys⁴,D-Phe⁷,Cys¹⁰)-α-MSH (4-13) (H-9220) |
| | (Met⁵,Pro⁶,D-Phe⁷,D-Trp⁹,Phe¹⁰)-α-MSH (5-13) (H-2716) |
| | (D-Trp⁷Ala⁸,D-Phe¹⁰)-α-MSH (6-11) amide (H-9990) |
| | Acetyl-(D-Lys¹¹,D-Val¹³)-α-MSH (11-13) (H-8615) |
| | Acetyl-(D-Val¹³)-α-MSH (11-13) (H-8610) |
| | (D-Pro¹²)-α-MSH (11-13) (free acid) (H-6590) |
| | MSH-B (H-3566) |
| | β-MSH (H-1475) |
| | Acetyl-(Cys¹¹,D-2-Nal¹⁴,Cys¹⁸)-β-MSH (11-22) amide (H-4352) |
| | (Deamino-Cys¹¹,D-2-Nal¹⁴,Cys¹⁸)-β-MSH (11-22) amide (H-4942) |
| | γ-MSH (3-8) (H-4335) |
| | γ1-MSH (H-4395) |
| | γ2-MSH (H-4400) |
| | Acetyl-(Lys⁰,Nle³)-γ2-MSH amide (H-5464) |
| | γ3-MSH (H-2922) |
| | δ-MSH (H-4405) |
| Morphine | Neuropeptide AF (H-4946) |
| Modulating | Neuropeptide FF (H-5655) |
| Neuropeptides | (D-Tyr1,N-Me-Phe³)-Neuropeptide FF (H-4752) |
| | Neuropeptide SF (H-4948) |
| | H-Pro-Gln-Arg-Phe-NH₂ (H-6865) |
| Natriuretic | Thr-Ala-Pro-Arg-Atrial Natriuretic Factor (1-28) (H-3046) |
| Peptides and | Atrial Natriuretic Factor (3-28) (H-1335) |
| Related Peptides | Atrial Natriuretic Factor (4-28) (H-1990) |
| | mini-ANP (H-3372) |
| | Prepro-Atrial Natriuretic Factor (26-55) (H-5472) (Cardiodilatin-Related Peptide) |
| | Prepro-Atrial Natriuretic Factor (56-92) (H-5474) |
| | Prepro-Atrial Natriuretic Factor (104-123) (H-3402) |
| | (Tyr0)-Prepro-Atrial Natriuretic Factor (104-123) (H-5516) |
| | Vasonatrin Peptide (VNP) (H-2502) |
| | Brain Natriuretic Peptide-26 (H-2948) |
| | Brain Natriuretic Peptide-32 (H-9060) |
| | (Tyr0)-Brain Natriuretic Peptide-32 (H-5698) |
| | Brain Natriuretic Peptide-32 (H-2952) |
| | Brain Natriuretic Peptide-34 (3-34) (H-5716) |
| | Brain Natriuretic Peptide-45 (H-8035) |
| | C-Type Natriuretic Peptide (1-53) (H-8420) |
| | C-Type Natriuretic Peptide (32-53) (H-1296) |
| | (Tyr⁰)-C-Type Natriuretic Peptide (32-53) (H-5518) |
| | Vasonatrin Peptide (VNP) (H-2502) |
| | Dendroapis Natriuretic Peptide(H-4904) |
| | (Des-Arg³⁰,Des-Pro³¹)-Dendroaspis Natriuretic Peptide(H-4888) |
| Neoendorphins | Dynorphin A (1-6) (H-2665) |
| | Leu-Enkephalin (H-2740) |
| | α-Neoendorphin (H-4410) |
| | α-Neoendorphin (1-8) (H-4415) |
| | β-Neoendorphin (H-4420) |
| Neurokinins | Neurokinin A (H-3745) |
| | (Tyr⁰)-Neurokinin A (H-9270) |
| | Neurokinin A (4-10) (H-5955) |
| | (β-Ala⁸)-Neurokinin A (4-10) (H-2786) |
| | (Nle¹⁰)-Neurokinin A (4-10) (H-9275) |
| | (Trp⁷,β-Ala⁸)-Neurokinin A (4-10) (H-2788) |
| | (Tyr⁵,D-Trp^{6,8,9},Arg-NH₂¹⁰)-Neurokinin A (4-10) (H-2072) |
| | Neurokinin B (H-2045) |
| | (N-Me-Phe⁷)-Neurokinin B (H-9280) |
| | (Pro⁷)-Neurokinin B (H-9285) |
| | (D-Pro²,D-Trp^{6,8},Nle¹⁰)-Neurokinin B (H-9290) |
| Neuromedins | GRP (18-27) (H-3120) |
| | Neuromedin B (H-3280) |
| | Neuromedin N (H-4150) |
| | Neuromedin U-25 (H-5538) |
| Neuropeptide Y | Neuropeptide Y (H-6375) |
| (NPY) | Biotinyl-Neuropeptide Y (H-5674) |
| | (Leu³¹,Pro³⁴)-Neuropeptide Y (H-3306) |
| | (D-Trp³²)-Neuropeptide Y (H-3312) |
| | (Tyr(Me)²¹)-Neuropeptide Y (H-3302) |
| | Neuropeptide Y (1-24) amide (H-3304) |
| | (Cys²)-Neuropeptide Y (1-4)-8-aminooctanoyl-(D-Cys²⁷)-Neuropeptide Y (25-32) |
| | (H-3298) |
| | Neuropeptide Y (2-36) (H-3316) |
| | Neuropeptide Y (3-36) (H-3326) |
| | Neuropeptide Y (13-36) (H-3324) |
| | (Leu31,Pro34)-Neuropeptide Y (13-36) (H-3318) |
| | Neuropeptide Y (18-36) (H-3296) |
| | Pancreatic Polypeptide (1-17)-(Ala³¹,Aib³²)-Neuropeptide Y (18-36) (H-5086) |
| | Neuropeptide Y (22-36) (H-9305) |
| | Tyr-Lys-Gly-(Cyclo(Glu²⁶,Lys²⁹),Pro³⁴)-Neuropeptide Y (25-36) (H-3972) |
| | (D-Tyr^{27,36},D-Thr³²)-Neuropeptide Y (27-36) (H-3328) |
| | ((Cys³¹,Nva³⁴)-Neuropeptide Y (27-36))2 (H-3704) |
| | (Pro³⁰,Tyr³²,Leu³⁴)-Neuropeptide Y (28-36) (H-3546) |
| | (His³²,Leu³⁴)-Neuropeptide Y (32-36) (H-3544) |
| | (Gly¹,Ser^{3,22},Gln^{4,34},Thr⁶,Ala¹⁹,Tyr²¹,Ala^{23,31},Aib³²)-Pancreatic Polypeptide (H- |
| | 5088) |
| Neurotensins | Neurotensin (H-4435) |
| | (Gln⁴)-Neurotensin (H-4460) |
| | (Trp¹¹)-Neurotensin (H-7130) |
| | (D-Trp¹¹)-Neurotensin (H-4475) |
| | (D-Tyr¹¹)-Neurotensin (H-4480) |
| | Neurotensin (1-6) (H-4440) |
| | Neurotensin (1-8) (H-4445) |
| | Neurotensin (1-11) (H-4455) |
| | Neurotensin (8-13) (H-1810) |
| | Acetyl-Neurotensin (8-13) (H-1020) |
| | (Dab⁹)-Neurotensin (8-13) (H-3404) |
| | (Lys⁸-(®)-Lys⁹)-Neurotensin (8-13) (H-8370) |
| | (Lys⁸,Lys⁹)-Neurotensin (8-13) (H-8380) |
| | (Lys⁹,Trp¹¹,Glu¹²)-Neurotensin (8-13) (Cyclic Analog) (H-2554) |
| | Neurotensin (9-13) (H-3830) |
| | (Boc-Lys⁹)-Neurotensin (9-13)-methyl ester (A-2590) |
| Nociceptins | Nociceptin (H-3036) |
| | Nociceptin (1-13) amide (H-4072) |
| | (Phe1-(®)-Gly2)-Nociceptin (1-13) amide (H-4564) |
| Orexins | Hypocretin (70-98) (H-5468) |
| | Orexin A (H-4172) |
| | Orexin B (H-4174) |
| Oxytocins | Carbetocin (H-5832) |
| | Oxytocin (H-2510) |
| | Oxytocin (free acid) (H-6885) |
| | Oxytocin-2-fluoroethyl amide (H-4236) |
| | (d(CH2)5¹,Tyr(Me)²,Orn⁸)-Oxytocin (H-4928) |
| | (Ile⁸)-Oxytocin (H-2505) |
| | (Phe²,Orn⁸)-Oxytocin (H-3178) |
| | (Ser⁴,Ile⁸)-Oxytocin (H-2520) |
| | (Thr⁴,Gly⁷)-Oxytocin (H-7710) |
| Pancreatic | Pancreatic Polypeptide (1-17)-(Ala³¹,Aib³²)-Neuropeptide Y (18-36) (H-5086) |
| Polypeptides | Pancreatic Polypeptide (H-1610) |
| | (Gly¹,Ser^{3,22},Gln^{4,34},Thr⁶,Ala¹⁹,Tyr²¹,Ala^{23,31},Aib³²)-Pancreatic Polypeptide (H- |
| | 5088) |
| | Pancreatic Polypeptide (31-36) (H-6895) |
| Peptide YY | Peptide YY (H-9180) |
| (PYY) | (Leu³¹,Pro³⁴)-Peptide YY (H-2812) |
| | (Pro³⁴)-Peptide YY (H-2808) |
| | Peptide YY (3-36) (H-8585) |
| Pituitary | PACAP-27 (H-1172) |
| Adenylate Cyclase | PACAP-27 (6-27) (H-8435) |
| Activating | PACAP-38 (H-8430) |
| Polypeptides | PACAP-38 (6-38) (H-2734) |
| (PACAP) | PACAP-38 (16-38) (H-5484) |
| | PACAP-38 (28-38) (H-5758) |
| | PACAP-38 (31-38) (H-5522) |
| Pneumadins | Pneumadin (H-8180) |
| Prolactin- | Prolactin-Releasing Peptide (1-31) (H-4382) |
| Releasing | Prolactin-Releasing Peptide (12-31) (H-4392) |
| Peptides | |
| Protein Kinase | Ac-Asp-Tyr(2-malonyl)-Val-Pro-Met-Leu-NH₂ (N-1485) |
| Related Peptides | Ac-Asp-Tyr(PO₃H₂)-Val-Pro-Met-Leu-NH₂ (N-1480) |
| | Ac-Ile-Tyr-Gly-Glu-Phe-NH₂ (M-2165) |
| | Ac-Ile-Tyr(PO₃H₂)-Gly-Glu-Phe-NH₂ (M-2170) |
| | Ac-Leu-Lys-Phe-Ser-Lys-Lys-Phe-OH (H-3224) |
| | Ac-Tyr(PO₃H₂)-Glu-Glu-Ile-Glu-OH (H-3724) |
| | H-Ala-Asp-Ala-Gln-His-Ala-Thr-Pro-Pro-Lys-Lys-Lys-Arg-Lys-Val-Glu-Asp- |
| | Pro-Lys-Asp-Phe-OH (H-3288) |
| | (Ala92)-Peptide 6 (H-3718) |
| | H-Ala-Pro-Arg-Thr-Pro-Gly-Gly-Arg-Arg-OH (H-3244) |
| | H-Arg-Arg-Arg-Ala-Asp-Asp-Ser-Asp-Asp-Asp-Asp-Asp-OH (H-2486) |
| | H-Arg-Arg-Glu-Glu-Glu-Thr-Glu-Glu-Glu-OH (H-3248) |
| | H-Arg-Arg-Leu-Ile-Glu-Asp-Ala-Glu-Tyr-Ala-Ala-Arg-Gly-OH (H-5445) |
| | H-Arg-Arg-Leu-Ile-Glu-Asp-Asn-Glu-Tyr-Thr-Ala-Arg-Gly-OH (H-1795) |
| | H-Arg-Arg-Lys-Asp-Leu-His-Asp-Asp-Glu-Glu-Asp-Glu-Ala-Met-Ser-Ile-Thr- |
| | Ala-OH (H-2484) |
| | H-Arg-Gly-Tyr-Ala-Leu-Gly-OH (M-1105) |
| | H-Arg-Lys-Arg-Ser-Arg-Ala-Glu-OH (H-3214) |
| | H-Arg-Lys-Arg-Thr-Leu-Arg-Arg-Leu-OH (M-1950) |
| | H-Arg-Lys-Ile-Ser-Ala-Ser-Glu-Phe-Asp-Arg-Pro-Leu-Arg-OH (H-3216) |
| | H-Asn-Pro-Glu-Tyr(PO₃H₂)-OH (H-2706) |
| | Autocamtide-2 (H-3218) |
| | Autocamtide-2-Related Inhibitory Peptide (H-3384) |
| | (Ala²⁸⁶)-Calmodulin-Dependent Protein Kinase II (281-302) (H-3246) |
| | Calmodulin-Dependent Protein Kinase II (281-309) (H-3254) |
| | Calmodulin-Dependent Protein Kinase II (290-309) (H-9365) |
| | cAMP-Dependent Protein Kinase Inhibitor-α (5-22) amide (H-3222) |
| | cAMP-Dependent Protein Kinase Inhibitor-α (5-24) (H-5950) |
| | Cyclo(-Gly-Tyr(PO₃H₂)-Val-Pro-Met-Leu) (H-2062) |
| | Ephrin-A2-Selective YSA-Peptide (H-5894) |
| | H-Gln-Arg-Arg-Gln-Arg-Lys-Ser-Arg-Arg-Thr-Ile-OH (H-9685) |
| | H-Gly-Arg-Gly-Leu-Ser-Leu-Ser-Arg-OH (H-7405) |
| | H-Gly-Ile-2-Nal-Trp-His-His-Tyr-OH (H-4084) |
| | (Cys⁰)-GTP-Binding Protein Gsa (28-42) (H-5788) |
| | H1-7 (H-1805) |
| | Kemptamide (M-2505) |
| | Kemptide (M-1510) |
| | (Trp⁴)-Kemptide (M-1525) |
| | (Val⁶,Ala⁷)-Kemptide (M-1515) |
| | H-Leu-Arg-Arg-Arg-Arg-Phe-D-Ala-Phe-Cys(NPys)-NH₂ (H-3696) |
| | H-Lys-Arg-Glu-Leu-Val-Glu-Pro-Leu-Thr-Pro-Ser-Gly-Glu-Ala-Pro-Asn-Gln- |
| | Ala-Leu-Leu-Arg-OH (H-3242) |
| | H-Lys-Arg-Thr-Leu-Arg-OH (M-1945) |
| | H-Lys-Lys-Arg-Ala-Ala-Arg-Ala-Thr-Ser-Asn-Val-Phe-Ala-NH₂ (H-3252) |
| | Malantide (H-3262) |
| | MAPKK2 (1-16) (H-5778) |
| | Myelin Basic Protein (4-14) (H-1072) |
| | Acetyl-(Gln⁴)-Myelin Basic Protein (4-14) (H-3238) |
| | Myristoyl-Arg-Lys-Arg-Thr-Leu-Arg-Arg-Leu-OH (N-1310) |
| | Myristoyl-Lys-Arg-Thr-Leu-Arg-OH (N-1305) |
| | Myristoyl-Phe-Ala-Arg-Lys-Gly-Ala-Leu-Arg-Gln-OH (N-1370) |
| | Neurogranin (28-43) (H-1554) |
| | p60 v-src (137-157) (H-8535) |
| | Peptide ε (H-3236) |
| | H-Phe-Ala-Arg-Lys-Gly-Ala-Leu-Arg-Gln-OH (N-1375) |
| | H-Phe-Lys-Lys-Ser-Phe-Lys-Leu-NH₂ (H-1638) |
| | Phosphorylase Kinase β-Subunit Fragment (420-436) (H-1968) |
| | PKI-tide (H-3234) |
| | pp60 c-src (521-533) (H-3256) |
| | pp60 c-src (521-533) (phosphorylated) (H-3258) |
| | H-Pro-Leu-Ser-Arg-Thr-Leu-Ser-Val-Ala-Ala-Lys-Lys-OH (H-9375) |
| | Protein Kinase C (19-31) (H-3232) |
| | (Ser25)-Protein Kinase C (19-31) (H-3286) |
| | Protein Kinase C (19-36) (H-9370) |
| | Protein Kinase C (530-558) (H-8045) |
| | S6 Phosphate Acceptor Peptide (H-9380) |
| | Syntide 2 (H-9385) |
| | H-Tyr-Ile-2-Nal-Gly-Lys(retro-Trp-His-His-H)-Phe-Lys-OH (H-4082) |
| | H-Tyr-Ile-Tyr-Gly-Ser-Phe-Lys-OH (H-2686) |
| | H-Tyr-Ser-Phe-Val-His-His-Gly-Phe-Phe-Asn-Phe-Arg-Val-Ser-Trp-Arg-Glu- |
| | Met-Leu-Ala-OH (H-3592) |
| | H-Val-Arg-Lys-Arg-Thr-Leu-Arg-Arg-Leu-OH (H-3284) |
| | VEGFR-KDR/Flk-1 Antagonist Peptide (H-5896) |
| Secretins | Secretin (H-3022) |
| | Secretin (5-27) (H-4940) |
| Somatostatins | Cortistatin-17 (H-5536) |
| | 3-Mercaptopropionyl-Tyr-D-Trp-Lys-Val-Cys-p-chloro-D-Phe-NH₂ (H-9505) |
| | 3-Mercaptopropionyl-Tyr-D-Trp-Lys-Val-Cys-Phe-NH₂ (H-8460) |
| | H-D-2-Nal-Cys-Tyr-D-Trp-Lys-Val-Cys-2-Nal-NH₂ (H-2126) |
| | Octreotide (H-5972) |
| | H-D-Phe-Cys-Tyr-D-Trp-Arg-Thr-Pen-Thr-NH₂ (H-3698) |
| | H-D-Pbc-Cys-Tyr-D-Trp-Orn-Thr-Pen-Thr-NH₂ (H-2186) |
| | Cyclo-Somatostatin (H-2485) |
| | Somatostatin-14 (H-1490) |
| | Somatostatin-14 (reduced) (H-4662) |
| | (Des-Ala¹,des-Gly²,His^{4,5},D-Trp⁸)-Somatostatin-14 (H-2495) |
| | (D-Phe⁷)-Somatostatin-14 (H-4664) |
| | (D-Ser¹³)-Somatostatin-14 (H-4666) |
| | (D-Trp⁸)-Somatostatin-14 (H-3198) |
| | (D-Trp⁸,D-Cys¹⁴)-Somatostatin-14 (H-1500) |
| | Tyr-Somatostatin-14 (H-4995) |
| | (Tyr¹)-Somatostatin-14 (H-5000) |
| | (Tyr¹¹)-Somatostatin-14 (H-1495) |
| | Somatostatin-14 (2-9) (H-4696) |
| | Somatostatin-14 (3-10) (H-4702) |
| | Somatostatin-14 (3-14) (H-4774) |
| | (D-Phe⁵,Cys^{6,11},N-Me-D-Trp⁸)-Somatostatin-14 (5-12) amide (H-5648) |
| | Somatostatin-14 (7-14) (H-4698) |
| | Somatostatin-25 (H-9580) |
| | Somatostatin-28 (H-4955) |
| | (Leu⁸,D-Trp²²,Tyr²⁵)-Somatostatin-28 (H-3202) |
| | Tyr-Somatostatin-28 (H-4990) |
| | Somatostatin-28 (1-12) (H-4945) |
| | Somatostatin-28 (1-14) (H-4950) |
| | (Tyr¹²)-Somatostatin-28 (1-14) (H-4960) |
| Substance P | Substance P (H-1890) |
| | Substance P (free acid) (H-1885) |
| | Substance P-methyl ester (H-1895) |
| | (p-Bz-Phe⁸)-Substance P (H-3334) |
| | (Nle11)-Substance P (H-1905) |
| | (Sar⁹,Met(O₂)¹¹)-Substance P (H-9410) |
| | (Tyr⁸)-Substance P (H-1915) |
| | Substance P (1-4) (H-1875) |
| | Substance P (1-7) (H-1582) |
| | Substance P (1-9) (H-1880) |
| | Substance P (2-11) (H-4775) |
| | Substance P (4-11) (H-4680) |
| | (D-Ala⁴)-Substance P (4-11) (H-2368) |
| | Substance P (5-11) (H-5726) |
| | (D-Glu⁵,D-Trp^{7,9,10})-Substance P (5-11) (H-2184) |
| | (Pyr⁵)-Substance P (5-11) (H-4875) |
| | (Pyr⁵,N-Me-Phe⁸,Sar⁹)-Substance P (5-11) (H-4880) |
| | Substance P (6-11) (H-5728) |
| | (Pyr⁶)-Substance P (6-11) (H-4920) |
| | (Pyr⁶,Pro⁹)-Substance P (6-11) (H-4925) |
| | Succinyl-(Asp⁶,N-Me-Phe⁸)-Substance P (6-11) (H-5600) |
| | Substance P (7-11) (H-4555) |
| | δ-Aminovaleryl-(Pro9,N-Me-Leu10)-Substance P (7-11) (H-3336) |
| | Substance P (9-11) (H-5880) |
| Syndyphalins | Syndyphalin SD-25 (H-5010) |
| | Syndyphalin SD-33 (H-5015) |
| Thymopoietins | H-Arg-Lys-Asp(Asp-Val-Tyr-OH)-Val-Tyr-OH (H-8690) |
| | Thymopentin (H-5805) |
| | Thymopoietin II (32-34) (H-8760) |
| | Thymopoietin II (32-35) (H-5915) |
| | Thymopoietin II (32-36)-ethyl ester (H-1034) |
| | Thymopoietin II (33-36) (H-2408) |
| | Thymopoietin II (34-36) (H-8765) |
| Thymosins | Thymosin α1 (H-6945) |
| | Thymosin β10 (H-2928) |
| | Thymosin β4 (16-38) (H-2926) |
| Thyrotropin- | Cyclo(-His-Pro) (G-1745) |
| Releasing | Prepro-TRH (178-199) (H-3598) (Corticotropin Release-Inhibiting Factor |
| Hormone (TRH) | (CRIF)) |
| | TRH (H-4915) |
| | TRH (free acid) (H-4910) |
| | (3,4-Dehydro-Pro-NH₂³)-TRH (H-4900) |
| | (Glu²)-TRH (H-2464) |
| | (His(1-Me)²)-TRH (H-7665) |
| | (Phe²)-TRH (H-2462) |
| | TRH-AMC (I-1440) |
| Thyrotropin- | TRH-Gly (H-1036) |
| Releasing | TRH-4MbNA (J-1380) |
| Hormone (TRH) | TRH-bNA (K-1490) |
| (cont.) | TRH-Potentiating Peptide (H-1434) |
| Tuftsins | H-Thr-Lys-Pro-Pro-Arg-OH (H-5045) |
| | Tuftsin (H-5035) |
| | (3,4-Dehydro-Pro³)-Tuftsin (H-8515) |
| | (Lys(Z)²)-Tuftsin (H-5025) |
| Urocortins | Stresscopin (H-5546) |
| | (Tyr⁰)-Stresscopin (H-5842) |
| | Stresscopin-Related Peptide (H-5548) |
| | (Tyr⁰)-Stresscopin-Related Peptide (H-5838) |
| | Urocortin (H-3722) |
| | Urocortin II (H-5852) |
| | Urocortin III (H-5634) |
| Valorphins | Valorphin (H-8670) |
| | Leu-Valorphin-Arg (H-8880) |
| Vasopressins | (1-Adamantaneacetyl1,D-Tyr(Et)²,Val⁴,Abu⁶,Arg^{8,9})-Vasopressin (H-7705) |
| | (Arg⁸)-Vasopressin (H-1780) |
| | (Arg⁸,des-Gly-NH₂⁹)-Vasopressin (H-3184) |
| | (4-(4-Azidophenyl)butyry¹¹,D-Tyr(Me)²,Arg⁶,Arg⁸,Tyr-NHz⁹)-Vasopressin (H- |
| | 3506) |
| | (3-(4-Azidophenyl)propiony¹¹,D-Tyr(Me)²,Arg⁶,Arg⁸,Tyr-NH₂⁹)-Vasopressin (H- |
| | 3434) |
| | (d(CH₂)₅¹,D-Ile²,Ile⁴,Arg⁸)-Vasopressin (H-2404) |
| | (d(CH₂)₅¹,D-Ile²,Ile⁴,Arg⁸,Ala-NH₂⁹)-Vasopressin (H-3056) |
| | (d(CH₂)₅¹,D-Phe²,Ile⁴,Ala-NH₂⁹)-Vasopressin (H-5506) |
| | (d(CH₂)₅¹,Tyr(Et)²,Val⁴,Arg⁸)-Vasopressin (H-7670) |
| | (d(CH₂)₅¹,D-Tyr(Et)²,Val⁴,Arg⁸,des-Gly⁹)-Vasopressin (H-3192) |
| | (d(CH₂)₅¹,Tyr(Et)²,Val⁴,Arg⁸,des-Gly⁹)-Vasopressin (H-3188) |
| | (d(CH₂)₅¹,Tyr(Et)²,Val⁴,Arg⁸,des-Gly-NH₂⁹)-Vasopressin (H-7690) |
| | (d(CH₂)₅¹,Tyr(Me)²,Arg⁸)-Vasopressin (H-5350) |
| | (d(CH₂)₅¹,D-Tyr(Me)²,Val⁴,Arg⁸)-Vasopressin (H-3182) |
| | (Deamino-Cys¹,D-Arg⁸)-Vasopressin (H-7675) (Desmopressin) |
| | (Deamino-Cys¹,D-Orn⁸)-Vasopressin (H-1064) |
| | (Deamino-Cys¹,b-(3-pyridyl)-D-Ala²,Arg⁸)-Vasopressin (H-3058) |
| | (Deamino-Cys¹,Val⁴,D-Arg⁸)-Vasopressin (H-3176) |
| | (Deamino-Pen¹,Tyr(Me)²,Arg⁸)-Vasopressin (H-5340) |
| | (Deamino-Pen¹,Val⁴,D-Arg⁸)-Vasopressin (H-5345) |
| | (3,5-Diiodo-Tyr²,Arg⁸)-Vasopressin (H-3638) |
| | (Lys⁸)-Vasopressin (H-2530) |
| | (Phenylac¹,D-Tyr(Et)²,Lys⁶,Arg⁸,des-Gly⁹)-Vasopressin (H-3186) |
| | (Phenylac¹,D-Tyr(Me)²,Arg^{6,8},Lys-NH₂⁹)-Vasopressin (H-1564) |
| | (Phenylac¹,D-Tyr(Me)²,Arg^{6,8},Tyr-NH₂⁹)-Vasopressin (H-3194) |
| | (Propionyl¹,D-Tyr(Et)²,Val⁴,Abu6,Arg^{8,9})-Vasopressin (H-9400) |
| | Val-Asp-(Arg⁸)-Vasopressin (H-5265) |
| | (Pyr⁴,Cys(H-Cys-OH)⁶,Arg⁸)-Vasopressin (4-8) (H-2456) |
| | (Arg⁸)-Vasopressin (4-9) (H-4092) |
| Vasoactive | PHM-27 (H-6355) |
| intestinal peptides | Prepro VIP (81-122) (H-6910) |
| (VIP) | Prepro VIP (111-122) (H-6915) |
| | Prepro VIP (156-170) (H-9190) |
| | VIP (H-3775) |
| | (Ala^{11,22,28})-VIP (H-5802) |
| | (D-4Cpa⁶,Leu¹⁷)-VIP (H-5515) |
| | (D-Phe²)-VIP (H-5640) |
| | VIP (6-28) (H-2066) |
| | VIP (10-28) (H-5205) |
| | (Pyr¹⁶)-VIP (16-28) (H-5635) |
| Miscellaneous | Buccalin (H-9235) |
| Peptides | Bursin (H-5920) |
| | Chromostatin (H-8475) |
| | Corticostatin (H-9045) |
| | Dermaseptin (H-1294) |
| | Diazepam Binding Inhibitor (DBI) (H-6760) |
| | Elcatonin (H-2247) |
| | Enterostatin (H-6405) |
| | Epidermal Mitosis Inhibiting Pentapeptide (H-6770) |
| | Follicular Gonadotropin-Releasing Peptide (H-6775) |
| | Gastric Inhibitory Polypeptide (H-5645) |
| | Granuliberin-R (H-6800) |
| | Seminal Plasma Inhibin (67-94) (H-1602) (also known as β-inhibin) |
| | Kentsin (H-3840) (also known as Contraceptive Tetrapeptide) |
| | Magainin I (H-6565) |
| | Magainin II (H-6570) |
| | Metorphamide (H-6855) |
| | β-Neuroprotectin (N-1340) |
| | Pancreastatin (33-49) (H-5905) |
| | Pancreastatin (H-6165) |
| | Proctolin (N-1015) |
| | Rigin (H-6920) |
| | Systemin (H-8675) |
| | Thyroid releasing hormone (TRH) (H-4915), (also known as Protirelin) |
| | Urotensin II (H-4768) |

The specific peptides listed for each class provided in Table 1 are intended to be exemplary. The invention can be applied as well to any peptide falling within the general classifications above (e.g., any peptide or peptide analog sharing affinity for the same molecular target and administered for the same therapeutic purpose(s)). For example, in addition to the interleukins recited in Table 1 interleukins include the IL-1 receptor antagonist and agonist peptides described in U.S. Pat. Nos. 5,861,476, 5,786,331, 5,880,096, 5,767,234, 5,608,035; the IL-2 receptor binding peptides described in U.S. Pat. No. 5,635,597; and the IL-5 binding peptides described in U.S. Pat. Nos. 5,668,110 and 5,654,276. Furthermore, in addition to the glucagon-like peptides recited in Table 1, glucagon-like peptides include synthetic analogs that reproduce many of the biological actions of GLP-1, but with a prolonged duration of action, such as liraglutide (also known as NN-2211, Novo Nordisk), CJC-1 131 (ConjuChem), LY315902 (Lilly), LY307161 (Lilly), and BIM51077 (Roche, Beaufour Ipsen) (see, for example, Holz et al., Curr. Med. Chem. 10:2471-83 (2003)). Glucagon-like peptides also include the peptides recited in U.S. Pat. Nos. 5,118,666, 5,120,712, 5,512,549, 5,545,618, 5,574,008, 5,614,492, 5,705,483, 5,958,909, 5,977,071, 5,981,488, 6,133,235, and 6,191,102, and the GLP-1 peptides recited in PCT publication No. WO 03/072195. In addition to the amylin peptides recited in Table 1, amylins include pramlintide (Amylin) (see, for example, Kruger et al., Drugs 64:1419-32 (2004)).

Preparation of Articles

The articles of the present invention may be formed in any shape desired. For example, the articles may be shaped to fit into a specific body cavity. They may also be formed into thin, flat disks, pellets, rods, or particles, such as microspheres. Alternatively, the articles may be shaped, then processed into the desired shape before use, or ground into fine particles. The desired shape of the article will depend on the specific application.

As used herein, the term “particles” includes, but is not limited to, microspheres. In a microsphere, a BAS is dispersed throughout the particle. The particles may have a smooth or irregular surface, and may be solid or slightly porous, but with a pore size smaller than the hydrodynamic radius of human growth hormone.

Preconditioning of the Biologically Active Substance

The particle size and distribution of the BAS can affect the release profile of the therapeutic articles. The particle size and distribution of the BAS can be adjusted using techniques known in the art, including the inclusion of additives, choice of equipment and methodology in the preparation of the articles, and processing conditions.

Desirably, the BAS is preconditioned to form of a microparticulate powder having a particle size of about 0.02 to 10 microns, 0.05 to 5 microns, or 0.1 to 4 microns, depending upon the route of administration for which they are being formulated.

The BAS can be preconditioned to a microparticulate powder using a variety of processes, including spray drying, flash freezing, crystallization, cryopelletization, precipitation, super-critical fluid evaporation, coacervation, homogenization, inclusion complexation, lyophilization, melting, mixing, molding, solvent dehydration, sonication, spheronization, spray chilling, spray congealing, spray drying, and combinations thereof. In some instances, appropriate additives can also be introduced to the BAS during preconditioning to facilitate the formation of a microparticluate powder. For example, such powders can be prepared by coating the surface of the particulate BAS particles with sugars, such as lactose, sucrose, trehalose, or dextrose; polysaccharides, such as maltodextrin or dextrates; starches; cellulose, such as microcrystalline cellulose or microcrystalline cellulose/sodium carboxymethyl cellulose; inorganics, such as dicalcium phosphate, hydroxyapitite, tricalcium phosphate, talc, or titania; polyols, such as mannitol, xylitol, sorbitol; or surfactants, such as PEG; or combinations thereof.

Alternatively, a microparticulate powder can be prepared from a suitable salt of the BAS. Acceptable salts include non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids; polymeric acids such as tannic acid, or carboxymethyl cellulose; and inorganic acid addition salts such as hydrochloric acid, hydrobromic acid, sulfuric acid, or phosphoric acid. Cationic salts can be prepared from zinc, iron, sodium, potassium, magnesium, meglumine, ammonium, and calcium, among others.

Typically, the final step of preconditioning involves preparing a finely divided powder by milling, micronizing, nanosizing, (e.g., under high pressure) or precipitating the BAS prior to its use in the macromer formulations described herein.

Polymerization of Macromers to Therapeutic Articles

The macromers of the present invention are polymerized using polymerization initiators under the influence of long wavelength ultraviolet light, visible light, thermal energy, or a redox system. In combination with the melt process of the invention, the use of long wavelength ultraviolet light is preferred.

Polymerization of the macromers may be initiated in situ by light having a wavelength of 320 nm or longer. When the polymerizable region contains acrylate groups, the initiator may be any of a number of suitable dyes, such as xanthine dyes, acridine dyes, thiazine dyes, phenazine dyes, camphorquinone dyes, acetophenone dyes, or eosin dyes with triethanolamine, 2,2-dimethyl-2-phenyl acetophenone, and 2-methoxy-2-phenyl acetophenone.

The polymerization may also take place in the absence of light. For example, the polymerization can be initiated with a redox system, using techniques known to those of skill in the art. In some cases it is advantageous to prepare articles using the methods described herein using a redox system, as radical initiator production occurs at reasonable rates over a wide range of temperatures.

Initiators that can be used in the redox system include, without limitation, peroxides such as acetyl, benzoyl, cumyl and t-butyl; hydroperoxides such as t-butyl and cumyl, peresters such as t-butyl perbenzoate; acyl alkylsulfonyl peroxides, dialkyl peroxydicarbonates, diperoxyketals, ketone peroxide, azo compounds such as 2,2′-azo(bis)isobutyronitrile (AIBN), disulfides, and tetrazenes.

Excipients

Excipients may be added to the melt prior to polymerization to, for example, modulate the hydrophobicity of the resulting article. Excipients that can be used in combination with the present invention include saccharides, such as of sucrose, trehalose, lactose, fructose, galactose, mannitol, dextran and glucose; poly alcohols, such as glycerol or sorbitol; proteins, such as albumin; hydrophobic molecules, such as oils; hydrophobic polymers, such as polylactic acid or polycaprolactone; and hydrophilic polymers, such as polyethylene glycol, among others. Excipients may also be incorporated during the preconditioning of the BAS. For example, a lipophilic salt of the BAS can be prepared (e.g., acrylamido-2-methyl-1-propanesulfonic acid), thereby altering the water solubility of the encapsulated BAS and its release profile.

The Melt Process

To prepare the articles described herin, the macromer is heated until it forms a melt. To the liquid macromer is added a) a BAS powder with or without preconditioning; b) a polymerization initiator dissolved in a minimal amount of solvent; and, optionally, c) additional excipients as desired to alter the release profile of the resulting therapeutic article. The resulting viscous liquid is a mixture containing suspended particles of BAS and ready for polymerization.

Prior to polymerization the melt can be formed into any desired shape as described above. For example, to form particles the viscous melt can be added to an immiscible liquid with vigorous mixing to form an emulsion and, for example, exposed to light to polymerize the macromers to form hydrogel particles incorporating the substance, such as a BAS. Typically, emulsion and polymerization is carried out under conditions in which the temperature is controlled to keep the macromer in a liquid state.

Non-miscible solvents that can be used to form an emulsion with the macromer-melt include, without limitation, silicon oil, mineral oil, polypropylene glycol, Migliyoyl 850, oils that are removed after production of the microspheres, and any oils generally regarded as safe (GRAS) by the Food and Drug Administration.

The microspheres prepared using the techniques described above are first washed to remove any oils used in emulsion methods, any organic solvents used in washing steps (e.g., to remove oils), and dried by lyophilization or by passing anhydrous gas (e.g., dry nitrogen) over or through a fluidized bed of the microspheres, so they have a long shelf life (without hydrolytic degradation) and the BAS remains biologically active. Prior to use for injectable formulations, the microspheres are reconstituted in a suitable solution, such as saline or other liquids. For pulmonary delivery, either freeze dried or reconstituted particles may be used.

Properties of the Therapeutic Articles

The articles of the present invention are biodegradable. Biodegradation occurs at the linkages within the extension oligomers and results in fragments which are non-toxic and easily removed from the body and/or are normal, safe chemical intermediates in the body. The articles have a high density of crosslinking in comparison articles produced by polymerization in solution having lower macromer content. These materials are particularly useful for the sustained delivery of low molecular weight BAS', since the tight crosslinking limit diffusion into and out of the articles prior to degradation. The relatively higher macromer content results in a much denser article, which swells in the body more slowly and, hence, degrades more slowly.

Use of the Therapeutic Articles

Macromers can be shaped into articles, for example, microspheres, and these articles are capable of degrading under in vivo conditions at rates that permit the controlled release of incorporated substances. Release of such a substance may occur by diffusion of the substance from the polymer prior to degradation and/or by diffusion of the material from the polymer as it degrades. Degradation of the polymer facilitates eventual controlled release of free macromolecules in vivo by gradual hydrolysis of the terminal degradable region. The burst effects that are sometimes associated with other release systems are thus avoided in a range of formulations.

The rate of release of a BAS depends on many factors, for example, the composition of the water soluble region, the degree of polymerization of the macromer. The rate of release of a BAS also depends on the rate of degradation of the degradable region of the macromer. For example, glycolic esters lead to very rapid degradation, lactic esters to somewhat slower degradation, and caprolactic esters to very slow degradation. When the degradable region consists of polyglycolic acid, the release period is less than one or two weeks. When the degradable region consists of poly(lactic acid), the release period is about one week or greater. When the degradable region consists of a copolymer of caprolactone and lactic acid or a copolymer of trimethylene carbonate and lactic acid, the release period is two weeks or greater. When the degradable region consists of poly(trimethylene carbonate) or a copolymer of caprolactone and trimethylene carbonate, the release period is about three weeks or greater. When the degradable region consists of poly(trimethylene carbonate) or poly(caprolactone), the release period is longer than about five weeks.

The precise rate of release of a BAS from an article can be further modified by altering the ratio of hydrophilic and hydrophobic components of the article. For example, a very soluble macromer will yield, after polymerization, a hydrophilic gel; hydrophilic hydrogels have been shown to degrade more rapidly than hydrophobic ones. A blend of a hydrophilic macromer (e.g., 4kL5) with a hydrophobic water insoluble macromer (3.4kC6) is used to form a polymerized hydrogel. This hydrogel will have a release rate that is in between the release rate of a hydrogel containing only lactic acid and a hydrogel containing only caprolactone. A macromer in which the degradable region is a copolymer of caprolactone and lactic acid will also have a release rate which is in between the release rate of a hydrogel containing only lactic acid and a hydrogel containing only caprolactone as the primary degradable group. Similarly, hydrophilicity of the active substance also affect the release rate of the BAS, with hydrophilic active substances generally released faster than hydrophobic substances.

Therapy

The polymer articles of the present invention may be used to treat a mammal, by delivering a BAS to the mammal. The articles may contain any BAS described herein, among others. Various routes of administration may be used to deliver the articles of the present invention, as described below.

The results of the treatment of an mammal with therapeutic articles containing a BAS, as described herein, will vary according to the BAS being delivered. For example, if Peptide YY (3-36) (see, for example, Korner et al., N. Engl. J. Med. 349(10):926 (2003)) is delivered through the therapeutic articles of the present invention, one would expect to observe an decrease in appetite as a result of such a treatment. If Dynorphin A (1-13) is delivered through the therapeutic articles, one would expect to observe a decrease in pain as a result of the treatment. If insulin is delivered through the therapeutic articles, then the treatment should result in a decrease in blood glucose levels.

The articles of the present invention provide optimal delivery of a BAS, because they release the BAS in a controlled manner with a low burst effect. The result of such a delivery rate is that the drug is delivered steadily over a desired period of time. A slower and steadier rate of delivery may in turn result in a reduction in the frequency with which the BAS must be administered to the mammal. In addition, a low burst effect may be highly desirable in some circumstances where the delivery of too much BAS to a site is deleterious to the mammal. It is also desirable where the peak levels obtained with subcutaneous administration produces a dose dependent side effect, such as nausea. Release from microparticles of the invention can maintain therapeutic levels without the resulting plasma peak levels associated with direct injection of the BAS and, hence, without the resulting side effect.

Routes of Administration of the Therapeutic Articles

Intramuscular and Subcutaneous Administration

The articles of the present invention can be used to administer microspheres that degrade over a day, several days, or even up to 3-6 months, by intramuscular injection or by subcutaneous injection.

For example, Dynorphin A (1-13) can be administered subcutaneously; the peptide leaves the microspheres at the site of injection as they degrade. Dynorphin A (1-13) enters the systemic circulation, where, in turn, it exerts its antinociceptive effects on the recipient.

For this application, particle sizes of up to 1 mm, or greater, can be used.

Intravenous Administration

Articles that contain a BAS useful in treating appetite, such as Peptide YY (to reduce appetite), neuropeptide Y, or agouti-related peptide (to stimulate appetite), can be administered by intravenous injection. The BAS is released over days to weeks. A therapeutic level of the BAS is maintained that results in a better clinical outcome. In addition, potentially lower total doses of a BAS can be administered, with a corresponding economic benefit. These approaches help promote patient compliance.

In the case of intravenous injection, it is important to formulate the microspheres in acceptable agents so the microspheres do not aggregate and clog blood vessels. The microspheres must be appropriately sized, so that they don't lodge in capillaries. For this application, particle sizes of 0.2-0.5 μm are preferred.

In a number of inflammatory conditions, as part of the inflammatory process that is mediated by selectin and ICAM expression/binding with neutrophil intravisation, blood vessels become leaky at the site of inflammation. Hydrogel microspheres may be administered; these microspheres will leak out of blood vessels at the site of inflammation, and then release their BAS payload locally over a period of time. Disease conditions where this approach may be useful could include, but are not limited to, inflammatory bowel diseases, asthma, rheumatoid arthritis, osteoarthritis, emphysema, and cystic fibrosis (with DNAase as the enzymatic drug).

Hydrogel microspheres that contain cytokines, lymphokines, or other compounds to treat cancer can be administered by intravenous injection. Blood vessels within large solid tumors are generally leaky, and the blood flow within them is often slow. Thus, microspheres could lodge within solid tumors and release their anticancer BAS locally, either killing tumor cells directly or by activating the immune system locally. This approach could be used, for example, with compounds such as interleukin 2, where the systemic and local toxicity has been dose limiting and where the resulting side effects are significant.

The microspheres of the present invention may be cleared relatively slowly from the circulation. Alternatively, the microspheres can be targeted to exit the circulatory system through leaky blood vessels or through more active targeting mechanisms, e.g., receptor mediated targeting mechanisms.

Oral Administration

In some portions of the gastrointestinal tract, there is relatively good transport of proteins across the intestinal mucosa into the systemic and local circulation. The articles of the invention, for example, freeze dried microspheres containing peptide (with very small particle sizes), can therefore be administered orally in an appropriate enteric formulation that protects the drug-containing microspheres from enzymatic attack and the low pH found in the upper GI tract. Such an enteric formulation could also be designed using several available technologies to gradually expel BAS-containing microspheres as the enteric capsule traverses the gastrointestinal tract. This is described in more detail in WO 99/03454 and in Mathiowitz et al., Nature 386:410 (1997). It is anticipated that this approach will have a number of advantages over other approaches for delivering proteins, peptides, and other molecules, even small molecules, orally. First, PEG is compatible with peptides and proteins, so the major manufacturing and stability problems found with other drug delivery approaches can be avoided. Secondly, dried hydrogels are very adhesive to wet tissue. The microparticles will bind well to the GI tract and will be transported into the system via the gastrointestinal circulation or release their contents on the intestinal mucosa; in turn, the drug will enter the systemic and gastrointestinal circulation. Chemical enhancers, or formulations containing compositions that utilize specific and non-specific biological transport mechanisms to facilitate transport across the GI tract into the systemic circulation, can be included as well.

Nasal Delivery

The articles of the present invention can also be used to administer compounds nasally. For example, a vaccine containing freeze dried or reconstituted microspheres can be administered nasally.

Inhalation

The use of the hydrogel particles of the invention can enhance the delivery of drugs to the lung. Administration to the lung provides for the delivery of drugs that can be transported across the lung tissue barriers and into circulation, as described WO 99/03454.

A problem with the delivery of active substances to the lung is that pulmonary macrophages can take up the materials, thus preventing the material from entering into systemic and local circulation. Uptake occurs when proteins adsorbed to the article's surface bind with receptors of the macrophages. To prevent uptake, the invention provides nonionic hydrogels, e.g., formed with polymers based on polyethylene glycol. These hydrogels adsorb low levels of proteins and thus bind poorly to cell surfaces. Anionic hydrogels, e.g., formed with polyacrylic acid, also adsorb relatively low levels of proteins and thus bind poorly to cell surfaces.

The methods and compositions of the invention can be used to form biocompatible microcapsules having a surface including water soluble non-ionic polymers, such as polyethylene oxide (PEO), to create resistance to cell adhesion, as described in U.S. Pat. No. 5,380,536, hereby incorporated by reference.

The size and density of the articles can also be selected to maximize the quantity of BAS that is delivered to the lung. For example, the macrophages will not take up large particles as efficiently as they will take up small particles. However, large particles are not delivered to the deep lung as well as small particles are. To overcome these conflicting factors, the invention provides small particles that can swell as they hydrate. The particles are administered to the deep lung as small (i.e., 1-5 μm), dry, or slightly wet, particles; upon hydration, they swell, and therefore become resistant to uptake by the pulmonary macrophages. The swelling can occur when the particles are hydrated from the dry state and when they are hydrated from one state of hydration to another by a change in temperature, pH, salt concentration, or the presence of other solvents, for example, depending upon the chemical and physical nature of the hydrogel polymer.

In addition to particles, the polymer may be provided in other shapes suitable for delivery to the deep lung. For example, PEG emulsion microspheres are subjected to high pressure and a vacuum onto a flat plate to form very light very thin layers, for example, having a snow flake consistency, that react differently to fluidic wind forces. The resulting thin flakes can be, e.g., 0.01 μm, 1 μm, or 10 μm thick.

The particles can be administered to the respiratory system alone, or in any appropriate pharmaceutically acceptable excipient, such as a liquid, for example, saline, or a powder. Aerosol dosages, formulations and delivery systems may be selected for a particular therapeutic application (see, for example, Gonda “Aerosols for delivery of therapeutic and diagnostic agents to the respiratory tract,” Critical Reviews in Therapeutic Drug Carrier Systems, 6:273 (1990); and “Aerosols in Medicine. Principles, Diagnosis and Therapy,” Moren, et al., Eds., Elsevier, Amsterdam, 1985).

Pulmonary drug delivery may be achieved using devices such as liquid nebulizers, aerosol-based metered dose inhalers, and dry powder dispersion devices. For the use of dry powder dispersion devices, the polymer particle incorporating the therapeutic agent is formulated as a dry powder, for example, by lyophilization or spray-drying. Methods for preparing spray-dried, pharmaceutical-based dry powders including a pharmaceutically acceptable amount of a therapeutic agent and a carrier are described in PCT WO 96/32149, hereby incorporated by reference.

Examples of a BAS that can be administered to the lung include, without limitation, insulin, antitrypsin, calcitonin, alpha interferon, beta interferon, GLP-1, and DNAse.

The following examples are put forth so as to provide those of ordinary skill in the art with a complete disclosure and description of how the methods and compounds claimed herein are performed, made, and evaluated, and are intended to be purely exemplary of the invention and are not intended to limit the scope of what the inventors regard as their invention.

### EXAMPLES

### Example 1

### Controlled Release Formulation of GLP-1

The process of making controlled release formulation of GLP-1 involves two steps, making a salt of the peptide and encapsulating the salt in a therapeutic article.

First, a GLP-1 salt was created using 2-acrylamido-2-methyl-1-propanesulfonic acid (AMPS). GLP-1 (between 25 and 50 mg) was dissolved in 1 mL 10 mM PBS buffer. The pH was adjusted to 5.5 by addition of AMPS (50 to 100 mg) until the GLP-1/AMPS salt precipitates from the solution. The solution was decanted and the precipitate lyophilized. The lyophilized GLP-1/AMPS salt was then used in the encapsulation procedure.

Second, 4.4kC5-A3 macromer (1 g) was weighed into a 15 mL centrifuge tube which was heated with a heating block at 50° C. until the macromer completely melted. 2,2-dimethaoxy 2-phenyl acetophenone (DMPA) in 1,4 dioxane (0.125 g of a 15% solution) was added to the melted macromer. This was followed by GLP-1/AMPS salt (50 mg) and the mixture was heated at 50° C. for 2-5 minutes until the contents turned into a viscous liquid. The viscous liquid was transferred into a 3-mL syringe and released into a solution of polypropylene glycol (PPG) forming an emulsion. During the process of emulsification, one can control the size of the particles by adjusting the flow rates of the oil and macromer phases. In this process, we used a rate of 25 mL/min for the PPG (oil) and 1 mL/min for the melted macromer liquid. The emulsion was collected in a beaker after flowing through two static mixers and then exposed to long wave ultra violet light (LWUV) for 1 hour to crosslink the macromer using radical polymerization. The resulting microspheres were washed with hexane and 10 mM citrate buffer at pH 6.0.

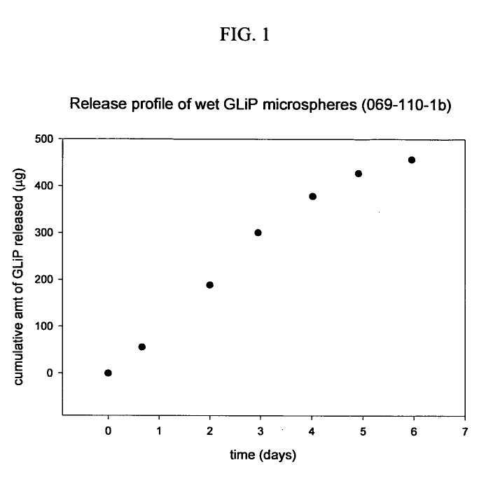

The microspheres were freeze-dried and tested in vitro using a fluidized bed column with 10 mM PBS buffer at pH 7.4 with a flow of 5 mL/day. The collected buffer was tested for GLP-1 using reverse phase column chromatography. The results are summarized in FIG. 1.

Therapeutic articles containing any BAS described herein can be formulated in a similar manner.

### Example 2

### Controlled Release Formulation of LH-RH

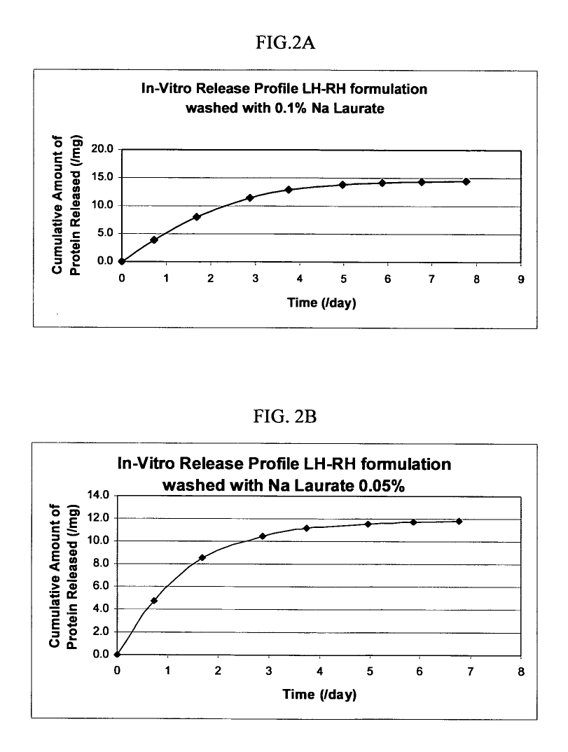

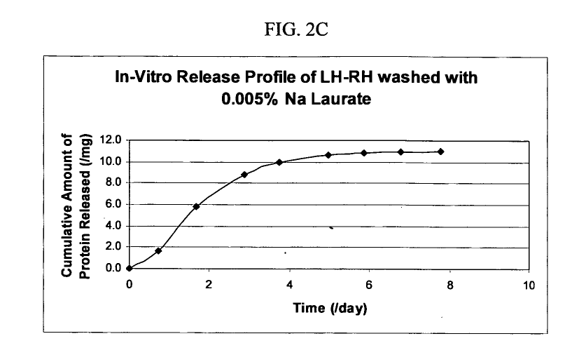

The macromer 4.4kC4-A3 (1 g) was heated to 50° C. and, once liquid, mixed with 0.15 g LH-RH, followed by the addition of 0.2 g of 10% DMPA solution in dioxane. The solution was emulsified with Migliyoyl 850. Once emulsified, the macromer was polymerized by exposure to long UV range lamp for a period of 1 hour. After the polymerization, the Migliyoyl 850 was removed by centrifugation, followed by washing with hexane. The hexane was removed from the microspheres by washing the microspheres with different concentrations of Sodium Laurate (0.1%, 0.05% and 0.005%) and monitored for in vitro release. The results are shown in FIGS. 2A, 2B, and 2C, respectively.

### Example 3

### Controlled Release Formulation of Fluticasone Propionate

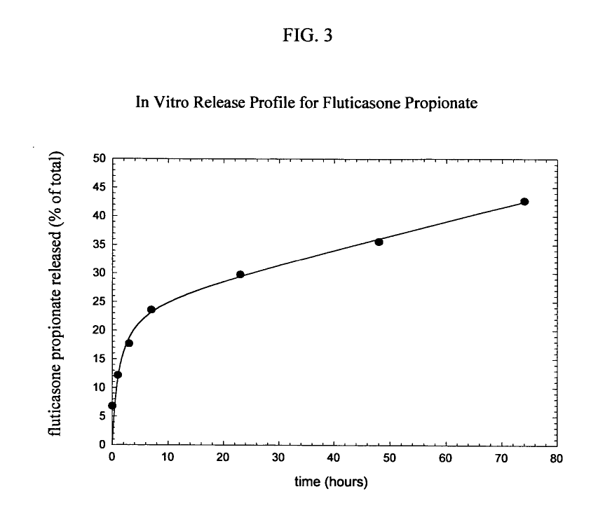

The macromer 4.4kC4-A3 (1 g) was heated to about 50° C. and, once liquid, mixed 0.1 g of 15% DMPA solution in dioxane. To this clear solution was added four tablets containing 250 micrograms fluticasone propionate each. The solution was mixed with polypropylene glycol to form an emulsion. Exposure to UV light for 1 hour polymerized the macromer, resulting in fluticasone propionate-containing microspheres. The microspheres were washed with hexane and sterile water followed by lyophilization. The microspheres were monitored for in vitro release. The results are provided in FIG. 3.

### Other Embodiments

All publications and patent applications, and patents mentioned in this specification are herein incorporated by reference.

While the invention has been described in connection with specific embodiments, it will be understood that it is capable of further modifications. Therefore, this application is intended to cover any variations, uses, or adaptations of the invention that follow, in general, the principles of the invention, including departures from the present disclosure that come within known or customary practice within the art.

Other embodiments are within the claims.

What we claim is:

1. A biocompatible therapeutic article comprising a biologically active substance within a polymerized macromer, the macromer comprising a poly(ethylene glycol) of between 1,000 and 12,000 daltons, at least one degradable polymer region which is hydrolyzable under in vivo conditions, and polymerized end groups, wherein the polymerized end groups are separated by at least one degradable polymer region and wherein said article when fully hydrated comprises at least 35% (w/w) polymerized macromer.

2. The article of claim 1, wherein said article when fully hydrated comprises less than 50% (w/w) water.

3. The article of claim 1, wherein said macromer comprises:
(a) a region forming a central core;
(b) at least two degradable regions attached to said core; and
(c) at least two polymerized end groups, wherein said polymerized end groups are attached to said degradable regions.

4. The article of claim 3, wherein said central core comprises a water soluble region consisting of a three-armed, four-armed, five-armed, six-armed, seven-armed, or eight-armed poly(ethylene glycol).

5. The article of claim 3, wherein said degradable regions comprise a polymer selected from the group consisting of poly(α-hydroxy acids), poly(lactones), poly(amino acids), poly(anhydrides), poly(orthoesters), poly(orthocarbonates), and poly(phosphoesters).

6. The article of claim 5, wherein said poly(α-hydroxy acid) is selected from the group consisting of poly(glycolic acid), poly(DL-lactic acid), and poly(L-lactic acid).

7. The article of claim 5, wherein said poly(lactone) is selected from the group consisting of poly(ε-caprolactone), poly(δ-valerolactone), and poly(γ-butyrolactone).

8. The article of claim 7, wherein said degradable regions comprise poly(caprolactone).

9. The article of claim 3, wherein said polymerized end groups are the product of a reaction between carbon-carbon double bonds capable of polymerizing said macromer.

10. The article of claim 3, wherein said macromer comprises:
(a) a water soluble region comprising three-armed poly(ethylene glycol);
(b) lactate groups attached to the region in (a); and
(c) acrylate groups capping the region in (b).

11. The article of claim 3, wherein said macromer is comprises:
(a) a water soluble region comprising three-armed poly(ethylene glycol);
(b) caprolactone groups on either side of region in (a); and
(c) acrylate groups capping either side of the region in (b).

12. The article of claim 1, wherein said biologically active substance is selected from peptides, carbohydrates, inorganic materials, antibiotics, antineoplastic agents, local anesthetics, antiangiogenic agents, vasoactive agents, anticoagulants, RNAi, antisense oligonucleotides, immunomodulators, cytotoxic agents, antiviral agents, antibodies, neurotransmitters, psychoactive drugs, oligonucleotides, proteins, lipids, and combinations thereof.

13. The article of claim 12, wherein said biologically active substance is a peptide.

14. The article of claim 13, said peptide is an opiod peptide or antimicrobial peptide.

15. The article of claim 12, wherein said peptide is selected from Acetelins, ACTH Peptides, Adrenomedullins, Amylins, Anti-HIV peptides, Anti-Inflammatory Peptides, Anti-Oxidant Peptides, Angiotensins, Apelins, BAM Peptides, Basic Fibroblast Growth Factor (FGF) Inhibitory Peptides, Bombesins, Bradykinins, Bradykinin-Potentiating Peptides (BPP), C3a and C3d Peptides, C5a-Related Peptides, Caerulein, Calcitonin and Calcitonin Precursors, Calcitonin Gene-Related Peptides (CGRP), Calpain Inhibitors, α-Casein Exorphins, β-Casomorphins, Cathepsin G Peptides, Cecropins, Ceratotoxins, Cerebellins, Cholecystokinin-Pancreozymin Peptides, Chorionic Gonadotropin (hCG) Peptides, CKS-17, Cocaine and Amphetamine Regulated Transcript (CART) Peptides, Conantokin G peptides, Corticotropin-Releasing Factor (CRF) and Analogs, C-Reactive Protein (CRP) Sequences, Defensins, Delta-Sleep Inducing Peptides (DSIP), Deltorphins, and Dermorphins, Eglin c peptides, Endomorphins, Endorphins, Endothelin Antagonists, Enkephalins and Proenkephalins, Farnesyltransferase Inhibitors, FIV Peptide, FMRFamide Peptides, Galanins and Galanin Message Associated Peptides (GMAP), Gastrins, Gastrin Releasing Peptides (GRP), Ghrelins, Glucagons and Glucagon-Like Peptides, Gluten Exorphins, GM-CSF Inhibitory Peptides, Growth Hormone-Releasing Factors (GRF) and Peptides (GHRP), Helodermins, Hirudins, Hylambatins, Insulin-like growth factors (IGF), Interleukins, Kinetensin s, Kyotorphins, Laminins, Leptins, Leucokinins, Leupeptins, Luteinizing hormone-releasing Hormone Peptides, Mastoparans, Melanin-Concentrating Hormones (MCH), Melanocyte-Stimulating Hormone-Release Inhibiting Factors (MIF-I), Melanotropin-Potentiating Factors (MPF), Motilins, Melanin-Stimulating Hormone (MSH) Peptides, Morphine Modulating Neuropeptides, Natriuretic Peptides and Related Peptides, Neoendorphins, Neurokinins, Neuromedins, Neuropeptide Y (NPY), Neurotensins, Nociceptins, Orexins, Oxytocins, Pancreatic Polypeptides, Peptide YY (PYY), Pituitary Adenylate Cyclase Activating Polypeptides (PACAP), Pneumadins, Prolactin-Releasing Peptides, Protein Kinase Related Peptides, Protein Kinase Related Peptides, Secretins, Somatostatins, Substance P, Syndyphalins, Thymopoietins, Thymosins, Thyrotropin-Releasing Hormone (TRH), Tuftsins, Urocortins, Valorphins, Vasopressins, Vasoactive intestinal peptides (VIP), collagenase-1 inhibitors, stromelysin-1 inhibitors, erythropoietin peptide agonists, follicle stimulating hormone antagonists, human neutrophil elastase inhibitors, kallikrein inhibitors, selectin binding peptides, exendins, exendin-4, and analogs thereof.

16. The article of claim 12, wherein said biologically active substance is a protein.

17. The article of claim 16, wherein said protein is selected from growth hormones, DNases, proteases, antibodies, poetins, cytokines, interferons, angiogenic factors, growth factors, and clotting factors.

18. The article of claim 16, wherein said protein is selected from human growth hormone, bovine growth hormone, urate oxidase, alronidase, alpha galactosidase, alpha glucosidase, trastuzumab, oprelvekin, muromonab-CD3, infliximab, abciximab, ritiximab, basiliximab, palivizumab, thymocyte globulin, cetuximab, daclizumab, erythropoietin, thrombopoietin, TNF-alpha, interferon alpha, interferon beta, vascular endothelial growth factor, endothelial cell growth factor, epidermal growth factor, basic fibroblast growth factor, and platelet derived growth factor, factor IV, factor VIII, factor VIIa, thyrotropin alfa, tissue plasminogen activator, glucocere-brosidase, etanercept, pegademase bovine, colony stimulating factor, follicle-stimulating hormone, luteinizing hormone, prolactin, relaxin, somatotropin-releasing hormones, tachykinins, thyroid-stimulating hormone, differentiation factors, colony-stimulating factors, ceredase, gibberellins, auxins, rhIGF-I/rhIGFBP-3 complex, and analogs thereof.

19. The article of claim 1, wherein the article releases 5% of the releasable biologically active substance from the article at a time greater than 1/16 of t₅₀.

20. The article of claim 1, wherein said biologically active substance has a molecular weight of less than 30,000 Daltons.

21. The article of claim 20, wherein said biologically active substance has a molecular weight of less than 10,000 Daltons.

22. The article of claim 1, wherein said article comprises at least 5% biologically active substance by dry weight.

23. The therapeutic article of claim 1, wherein said article is formed by a method comprising the following steps:
(a) heating the macromer until it melts;
(b) forming a mixture of biologically active substance and melted macromer; and
(c) polymerizing the mixture to form said article.

24. The article of claim 23, wherein the method further comprises the step of forming particles of said article.

25. A method for making a controlled release therapeutic article for delivery of a biologically active substance, said article comprising a biologically active substance within a polymerized macromer, the macromer comprising at least one water soluble polymer region, at least one degradable polymer region which is hydrolyzable under in vivo conditions, and polymerized end groups, wherein the polymerized end groups are separated by at least one degradable polymer region, said method comprising the steps of:
(a) heating the macromer until it melts;
(b) forming a mixture of biologically active substance and melted macromer; and
(c) polymerizing the mixture to form said therapeutic article.

26. The method of claim 25, wherein the mixture of step (b) is emulsified prior to step (c).

27. The method of claim 25, wherein the mixture of step (b) comprises a biologically active substance in the form of particles having a mean size of 0.02 to 10 microns.

28. The method of claim 25, wherein said article when fully hydrated comprises at least 35% (w/w) polymerized macromer.

29. The method of claim 25, wherein said article when fully hydrated comprises less than 50% (w/w) water.

30. The method of claim 25, wherein said biologically active substance has a molecular weight of less than 30,000 Daltons.

31. The method of claim 30, wherein said biologically active substance has a molecular weight of less than 10,000 Daltons.

32. The method of claim 31, wherein said biologically active substance has a molecular weight of less than 5,000 Daltons.

33. A method for delivering a biologically active substance to a mammal, said method comprising administering the article of claim 1 to said mammal.

34. The method of claim 33, wherein said article is administered to the lung of said mammal.

35. The method of claim 33, wherein said article is administered intravenously.

36. The method of claim 33, wherein said article is administered subcutaneously.

37. The method of claim 33, wherein said article is administered intramuscularly.

38. The method of claim 33, wherein said article is administered orally.

39. The method of claim 33, wherein said article is administered nasally.

40. The method of claim 33, wherein said mammal is a human.

41. The method of claim 33, wherein said biologically active substance is selected from peptides, carbohydrates, inorganic materials, antibiotics, antineoplastic agents, local anesthetics, antiangiogenic agents, vasoactive agents, anticoagulants, immunomodulators, cytotoxic agents, antiviral agents, antibodies, neurotransmitters, psychoactive drugs, oligonucleotides, proteins, lipids, and combinations thereof.

42. The article of claim 1, wherein said biologically active substance is parathyroid hormone or an analog thereof.

43. The article of claim 1, wherein said biologically active substance is etanercept or an analog thereof.

44. The article of claim 1, wherein said biologically active substance is epoetin or an analog thereof.

45. The article of claim 1, wherein said biologically active substance is filgrastim or an analog thereof.
